(19) United States
(12) Patent Application Publication
(10) Pub. No.: US 2023/0124918 A1
(43) Pub. Date: Apr. 20, 2023

(54) ADJUSTABLE AIRWAY STABILIZATION SYSTEM FOR PATIENT FACIAL GEOMETRIES OF VARIOUS SIZES AND FOR PEDIATRIC AND NEONATAL APPLICATIONS

(71) Applicant: Securisyn Medical, LLC, Littleton, CO (US)

(72) Inventors: Arthur Kanowitz, Littleton, CO (US); Janis Paulis Skujins, Delano, MN (US); Patrick Boldenow, Minneapolis, MN (US); Nicholas Rydberg, Stillwater, MN (US); Mark Bruning, Monument, CO (US); Katherine McIntyre, Denver, CO (US); Nam Trinh, Highlands Ranch, CO (US)

(73) Assignee: Securisyn Medical, LLC, Littleton, CO (US)

(21) Appl. No.: 17/909,364

(22) PCT Filed: Mar. 9, 2021
(86) PCT No.: PCT/US2021/021592

§ 371 Date: Sep. 3, 2022

Related U.S. Application Data
(60) Provisional application No. 62/987,068, filed on Mar. 9, 2020.

(51) Int. Cl. A61M 16/04

(52) U.S. Cl. CPC A61M 16/0497; A61M 16/0465

(57) ABSTRACT

An airway. stabilization system that may be used with human patients or with animal patients in veterinary applications having anatomical and facial geometries of various sizes and configurations including pediatric, and, in particular, addresses the unique challenges associated with maintaining the mechanical ventilation of infants and children. The stabilization system may be fitted to any airway device or endotracheal tube apparatus of any size to maintain an airway in a human or animal patient’s trachea and allows both lateral and longitudinal adjustment of the airway device insertion depth and prevents unintended extubation of a patient resulting from the application of multidirectional forces of any type to the airway device.

### CROSS REFERENCE TO RELATED APPLICATIONS

This application claims priority to U.S. Provisional Pat. Application No. 62/987,068 filed Mar. 9, 2020, the entire disclosure of which is incorporated herein by reference.

### FIELD OF THE INVENTION

The present invention relates generally to human and veterinary medical devices. Specifically, the present invention relates to an airway stabilization system designed to maintain an airway device in a preselected position in the trachea of a human patient or an animal and for preventing clinically significant movement thereof and unintentional extubation of the patient or animal in response to the application of significant multidirectional forces to the airway device. More specifically, the system of the present invention relates to an adjustable airway stabilization system and securing device that enables precise, safe and effective positioning of an airway device or endotracheal tube apparatus (ETT) in an airway of patients having facial geometries of various sizes, including pediatric patients.

### BACKGROUND OF THE INVENTION

Endotracheal intubation is a medical procedure used to place an airway device (artificial airway) into a patient’s trachea or airway. The use of an airway device is mandated in situations where an individual, or an animal in veterinary applications, is unable to independently sustain the natural breathing function or maintain an open airway due to unconsciousness, trauma, disease, drugs or anesthesia. Thus, life-saving mechanical ventilation is provided through the airway device, which may be in the form of an endotracheal tube (ETT), or a supraglottic airway device such as a laryngeal mask airway (LMA), King Airway, or one of several other commercially available airway devices.

Endotracheal intubation is accomplished by inserting an airway device into the mouth, down through the throat and larynx, and into the trachea. This procedure creates an artificial passageway through which air can freely and continuously flow in and out of a patient’s lungs and prevents the patient’s airway from collapsing or occluding.

It is very important that the airway device be positioned correctly and maintained in the correct position in the trachea. If the device moves out of its proper position in the trachea and into the right main stem bronchial tube, only one lung will be ventilated. Failure to ventilate the other lung can lead to a host of severe pulmonary complications. Moreover, if the airway device moves completely out of the trachea and into the pharynx, esophagus or completely outside the body, the patient will become hypoxic due to the lack of ventilation to the lungs, a condition which typically results in life-threatening brain injury or death within a matter of only a few minutes.

Even after an airway device has been positioned correctly, subsequent movement of the patient can lead to inadvertent movement of the device, as hereinabove described An intubated patient may restlessly move about and may also attempt to forcibly remove an airway device, whether conscious or subconscious, particularly if the patient is uncomfortable or having difficulty breathing, which can lead to panic. In the case of an animal patient, agitation may be particularly pronounced due to the animal’s lack of cognitive awareness or understanding of its circumstances and an instinctual survival fight or flight response. A large animal or a carnivore can pose a serious danger not only to itself but also to a treating veterinarian and anyone in close proximity under such circumstances.

Medical emergencies may occur anywhere. Accordingly, emergency medical service personal (i.e., paramedics) may be called upon to insert airway devices in out-of-hospital emergency settings, for example at accident scenes, and military personnel in combat situations, in emergency response vehicles, as well as in hospital settings by emergency department physicians, anesthesiologists, and critical care clinicians. Therefore, such unintentional movement of either the patient or an airway device is not uncommon, particularly when the patient is moved from an out-of-hospital setting, such as any one of the afore-mentioned scenarios, to an emergency department of a hospital. Further, anytime an intubated patient is be moved, for example, not only from an ambulance to a trauma facility, but also from one hospital to another hospital, from one area of the hospital to another area in the same hospital (imaging, laboratory, operating theater), or from a hospital to an outpatient rehabilitation facility, unintentional movement of an airway device is a risk. Even repositioning an intubated patient in a hospital bed, or in the case of an animal, in a recovery cage, may cause unintentional movement of the endotracheal tube.

Inadvertent movement of an airway device may also occur as a result of moving external ventilation equipment, such as a conventional mechanical ventilator or bag valve mask. Typically, the external ventilation equipment is connected to the external end of the device by an air conduit to establish air flow to and from the lungs. Inadvertent pulling on, or other excessive movement of the air conduit, may not only disconnect it from the airway device, but may also transfer movement to the airway device, thereby shifting it from its proper position and causing unplanned extubation.

Unplanned extubation is a hazardous and costly problem in humans, a problem which studies have established occurs at an unacceptably high rate. For example, Statistics published by the Society for Critical Care Medicine states that in 2017 there were 1.65 Million intubated, mechanically ventilated ICU patients in the United States (Medicine, S.f.C.C. Critical Care Statistics 2017). A review of the world-wide medical literature suggests that the world-wide rate of unplanned extubation averages approximately 7.31% of extubated patients. Lucas de Silva, Unplanned Endotracheal Extubation in the Intensive Care Unit: Systematic Review, Critical Appraisal, and Evidence-Based Recommendations. Anesth Analg 2012; 114:1003-14. Applying the world-wide average to the U.S. figure above, an estimated 120,000 patients in the United States alone experience an unplanned extubation each year. Such unplanned extubations are costly, not only for patients who experience increased rates of morbidity and mortality, but also for hospitals, physicians and insurance companies who incur the liability costs associated therewith. The annual intensive care unit (ICU) bed cost associated with unplanned extubations in the United States alone is estimated at $4.9 Billion, which includes imaging, pharmacy, and laboratory expenses. (Extrapolated using data from the Carson study referenced above and the cost of long-term care according to the U.S. Department of Health and Human Services National Clearinghouse for long-term care information. See also S.K. Epstein, M.L. Nevins & J. Chung, Effect of Unplanned Extubation on Outcome of Mechanical Ventilation, Am. Journal of Respiratory and Critical Care Medicine, 161: 1912-1916 (2000) which discusses the increased likelihood of long-term care outcome). Moreover, it is not unknown for jury damage awards in personal injury lawsuits arising from unplanned extubations to be in excess of $35 M.

Clearly, the economic losses related to unintentional extubation of animals are not as serious as the well-documented economic losses in human cases. Nonetheless: economic losses in the agricultural sector of valuable farm animals, breeding stock, and food resources, particularly in underdeveloped countries, cannot be ignored. On the domestic side, as anyone who has lost a beloved pet can attest, the emotional pain can equal that experienced at the loss of a family member. In view of the foregoing, the high incidence of unplanned extubation and the associated increase in healthcare costs implies that an improved restraining system is sorely needed, a system which has the capacity to resist the application of forces which would otherwise result in movement of the airway device.

Various prior art systems have attempted to address the problem of maintaining an airway device in the correct position and preventing unintentional extubation. The most common approach for securing an airway device (typically, an endotracheal tube) is with adhesive tape. Umbilical tape may be used as an alternative. Both present the same challenges. The tape is tied around the patient’s neck and then wrapped and tied around the smooth outside surface of the endotracheal tube itself. Arranged in this fashion, the tape is intended to anchor the endotracheal tube to the corner of the patient’s mouth and prevent its unintentional movement. While the use of tape in this manner provides some benefit, the restraint available from the tape usually diminishes because the tape becomes covered and/or saturated with blood, saliva, or other bodily fluids. Consequently, the endotracheal tube may be readily moved from its preferred position in a patient’s trachea. In spite of its widespread use, adhesive or surgical tape is woefully inadequate in providing protection against movement resulting from the application of multidirectional forces such as bending, torsional/rotational or substantial lateral forces to the device, forces which may exceed fifty (50) pounds in magnitude.

The results of two studies of the restraint capabilities of current devices and methods are set forth in Tables 1 and 2 below. Such devices and methods do not provide sufficient resistance to prevent unplanned extubation. Clinically significant movement is defined as longitudinal movement of the airway device in a direction towards or away from the patient’s mouth to a point where the tip of the airway device has moved beyond the larynx or vocal cords. Typically, such movement in a human patient is in the range of five (5) to seven (7) centimeters. In an animal, it may be significantly more or less, depending upon the size of the animal. For example, clinically significant movement in a cat is considerably less than clinically significant movement in a long-necked animal such as a horse or a giraffe.

TABLE 1

Restraint Capabilities of Current Devices and Methods in Human Applications

| | Median | Min | Max |
|---|---|---|---|
| Thomas Tube Holder | 12.98 | 2.64 | 22.44 |
| Adhesive Tape | 19.58 | 3.96 | 39.6 |
| Non-Adhesive Tape | 7.48 | 2.42 | 27.72 |

Force to Extubate (7 cm movement) in Lbs. Owens, et al. Resuscitation (2009)

TABLE 2

| | Median | Min | Max |
|---|---|---|---|
| Adhesive Tape (Lillehei) | 19.5 | 15 | 25 |
| Tube Tamer | 12.9 | 10 | 15 |
| Precision Medical | 8.6 | 7 | 10 |
| Biomedix Endogrip | 10.7 | 6 | 12 |
| Thomas Tube Holder | 37 | 28 | 43 |

Force to Extubate (2 cm movement) in Lbs. Carlson, et al. Annals of Emergency Medicine 2007

In the human medical field, efforts to address the foregoing problems have resulted in apparatus such as disclosed in U.S. Pat. No. 5,353,787 issued Oct. 11, 1994, to Price. Price discloses an apparatus having an oral airway for providing fluid communication for the passage of gas from a patient’s mouth through his or her throat and into the trachea, the oral airway being releasably attached to an endotracheal tube for useorce to Extubate (2 cm movement) in Lbs. Carlson, et al. Annals of E in combination therewith. While Price’s apparatus eliminates the smooth surface of the tube and resists longitudinal movement in relation to the oral airway, his system does not address the above-identified problem of resisting multidirectional forces. Moreover, Price’s device cannot prevent clinically significant movement of an airway device in relation to the vocal cords and an unplanned extubation resulting therefrom.

Other attempts to solve the aforementioned problems have employed auxiliary mechanical securing devices to maintain the position of an endotracheal tube in a patient. Many of these auxiliary mechanical devices include some type of plate which is attached to the patient’s face, usually with one or more straps that extend around the back of the patient’s head or neck. The faceplate includes some type of mechanical contact device that grips the smooth surface of the endotracheal tube. Typical mechanical contact devices include thumb screws, clamps, adhesives, locking teeth, and straps. By way of example, U.S. Pat. No. 4,832,019 issued to Weinstein et al. on May 23, 1989, discloses an endotracheal tube holder which includes a hexagonally shaped gripping jaw that clamps around the tube after it has been inserted into a patient’s mouth and a ratchet-type locking arrangement designed to retain the gripping jaw in position around the tube. Weinstein’s patent disclosure states specifically that the tube will not be deformed. However, the fundamental mechanics of a hexagonal receptacle applied around a cylindrical tube to stabilize it reveal that the hexagonal structure will not impart force to the tube of sufficient magnitude to prevent longitudinal movement. It has been found that if sufficient pressure is applied directly to the tube by the gripping jaw, the tube will deform or even crush, thereby decreasing ventilatory efficiency to the point that airflow to the patient’s lungs will be restricted or even cut off, an extremely serious problem.

U.S. Pat. No. 7,568,484 issued on Aug. 4, 2009, and U.S. Pat. No. 7,628,154 issued on Dec. 8, 2009, both to Bierman et al., disclose endotracheal tube securement systems which include straps extending from the corners of a patient’s mouth above and below the patient’s ears on each side of the patient’s head. However, the devices disclosed in the ‘484 and the ‘154 patents merely retain the position of the tube in the patient’s mouth and cannot prevent movement thereof in various directions, either longitudinally, rotationally or laterally, as hereinabove described.

Specifically, to maintain an effective restraint, attending medical personnel increase the amount of clamping force applied on an airway device. Increasing the amount of clamping force to an effective level may pinch the device to the point where a portion of the inner tube diameter (and hence air passageway) is significantly restricted. Restricting the cross-sectional size of the air passageway decreases the ventilatory efficiency of the tube, thereby decreasing the respiratory airflow. The restriction of the cross-sectional size of the air passageway creates resistance to both inspiratory airflow and expiratory airflow. Insufficient airflow during inspiration can lead to hypoxemia, which is problematic, but can be overcome by increasing the positive pressure of the ventilation source. However, during expiration, any increased pressure due to constriction or decreased tube diameter, increases the amount of work a patient must perform to simply exhale, increased pressure can also lead to barotrauma in the lungs and resistance to expiratory airflow can lead to multiple other adverse effects within the lungs. Impairing a patient’s ventilations may result in serious medical effects, particularly with patients whose functions are already compromised. Therefore, the ability for clinicians to adequately stabilize an airway device for prevention of unplanned extubation without constriction of the air passageway is crucial for patient safety.

In addition to the foregoing, other issues have arisen with respect to standard respiratory connectors that serve as conduits between the endotracheal tube and artificial ventilator for the purpose of maintaining a continuous flow of air from the ventilation source to the patient’s lungs. Standard connectors must be tightly seated into the endotracheal tube to avoid unintentional disconnection of the ventilation source from the endotracheal tube during mechanical breathing. A tightly seated connector is often difficult for the clinician to remove from the endotracheal tube, when necessary. Therefore, an airway device with a connector that prevents unintentional disconnection yet allows for quick and easy intentional connection and disconnection is needed.

More recently, U.S. Pat. No. 8,001,969 issued on Aug. 23, 2011, and U.S. Pat. No. 8,739,795 issued on Jun. 3, 2014, both to Arthur Kanowitz, the inventor of the present invention, disclose airway stabilization systems which address many of the problems set forth above. Continuing research into ways of providing even more advanced and rapidly deployable airway stabilization systems have resulted in yet further improvements to the overall design of the system components, which also may be used to address analogous problems in the veterinary medical field. However, issues related to the wide range of ETT tube sizes and patient facial geometries remain unaddressed.

Unplanned and accidental extubation of children and neonates is an area of significant concern. Infants and children have unique challenges with endotracheal tube securement, and pediatric patients are at particularly high risk for unplanned extubation due to anatomic and physiologic factors. Securement of endotracheal tubes is vital to the successful treatment and even the survival of premature infants who frequently require months of intubation in pediatric intensive care units. Current practice employs tape which is wrapped around the tubular body of an ETT and then around the infant’s head and/or neck. However, tape if frequently found to be irritating to the highly sensitive skin of infants, and a tape-free airway device securement system is needed to ensure reliable mechanical ventilation. Unplanned extubations in newborns and pediatric patients are unfortunately common, potentially devastating, and costly, often leading to a variety of serious, life-threatening cardiovascular and respiratory complications such as hypoxia, hypercarbia, airway trauma, ventilator associated pneumonia, intraventricular hemorrhage, and death. Intubation systems presently available for mechanical ventilation of more fully developed children and adult patients are simply unsuitable for intubation of young children and infants. An improved pediatric securement system that reduces the rate of unplanned extubation in infants and children is needed which would improve outcomes for these categories of patients.

In view of the foregoing, it will be apparent to those skilled in the art from this disclosure that a need exists for an improved airway stabilization system which not only protects an airway device from occlusion and crushing, but also is easier to apply to a patient while at the same time maintains the device in its preferred position in a patient’s trachea and prevents clinically significant movement thereof with respect to the vocal cords as a result of the application of multidirectional forces of significant magnitude thereto. Specific needs exist to address the variations effective positioning of an airway device or endotracheal tube apparatus (ETT) in an airway of patients having anatomical and facial geometries of various sizes and to address the unique challenges associated with maintaining the mechanical ventilation of infants and children. The present invention addresses these needs in the art as well as other needs, all of which will become apparent to those skilled in the art from the accompanying disclosure.

### SUMMARY OF THE INVENTION

In order to address the aforementioned needs in the art, a complete airway stabilization system is provided which may be fitted to any airway device that may be used with human patients or with animal patients in veterinary applications to maintain an airway in a human or animal patient’s trachea, the patient having a head, a face, a chin, a chest, a mouth, an oral cavity, vocal cords or larynx, a thoracic area, a trachea having a length and forming an airway in the patient, and a carina defining a point at which the trachea separates into a left and a right bronchial tube. The stabilization system prevents clinically significant movement of the airway device with respect to a patient’s vocal cords in response to the application of forces in any direction to the device, be they longitudinal, torsional/rotational or bending.

The airway device has a flexible elongate body which conforms to a patient’s trachea after it is installed in the patient. The airway device includes a continuous sidewall having outer and inner surfaces extending between a proximal (patient-end) and a distal (machine-end) portion thereof, thereby forming a hollow conduit through which the airway is established.

In an embodiment, a securing apparatus or stabilizer includes a frame, bridge or support member, as the terms will be used interchangeably herein, secured to the patient and a tower structure or clamshell-type clamping member operatively connected thereto, as the elements and operation of which are described in greater detail below. The clamping member is configured to interact in clamping engagement with the continuous sidewall of the airway device via a carriage member or collar adjustably positioned in the tower structure to prevent clinically significant movement of the patient end of the airway device with respect to the vocal cords of the patient. The bridge or support member is formed of a single structure to allow greater ease of application, the bridge or support member being structured and arranged to be secured over the face of a patient and operatively connected to the clamping member while, at the same time, providing ease of access to the patient’s oral cavity for suctioning and oral hygiene.

The clamping member or tower structure is adjustably secured to the frame or support member and extends outwardly therefrom along a longitudinal axis in a direction away from a patient’s face. The clamping member includes a pair of oppositely disposed pivotally interconnected c-shaped collars or clamshells, each collar or clamshell having a first end and a second end and a body portion extending therebetween, the body portion having an inner surface and an outer surface, the inner surface of at least one of the c-shaped collars or clamshells including a plurality of substantially uniformly spaced-apart annular flanges positioned axially along the inner surface of the body portion and extending substantially inwardly therefrom, and a plurality of structural recesses positioned axially along the inner surface of the body portion intermediate an adjacent two of the plurality of substantially uniformly spaced-apart annular flanges and structural recesses of the clamshells.

In one embodiment, the carriage member includes a pair of pivotally interconnected elongate c-shaped cylindrical members, each positioned within and operatively connected to a respective one of the c-shaped collars or clamshells and extending outwardly from the patient’s face coaxially with the longitudinal axis of the clamping member or tower structure. Each of the elongate cylindrical members includes first and second ends and a body portion having an inner surface and an outer surface extending therebetween. The outer surface of at least one of the pair of cylindrical members includes at least one annular flange and structural recess extending radially outwardly from the outer surface and adapted to operatively interact with one of the plurality of structural recesses formed intermediate the substantially uniformly spaced-apart annular flanges positioned axially along the inner surface of the at least one of the clamshells to retain the airway device in a preselected position in a patient’s airway. The inner surface may be coated with an adhesive material, by way of example and not of limitation, a pressure sensitive adhesive (PSA) adapted to adhesively engage the outer surface of an airway device.

In another embodiment, the carriage member comprises one or more semi-cylindrically or c-shaped grommets each positioned within and operatively connected to a respective one of the c-shaped collars or clamshells and extending outwardly from the patient’s face coaxially with the longitudinal axis of the clamping member. Similar in configuration to the clamshells, each c-shaped grommet has first and second ends and a body portion having an inner surface and an outer surface extending therebetween. The outer surface of each grommet includes at least one annular flange and structural recess extending radially outwardly from the outer surface and adapted to operatively interact with one of the plurality of structural recesses formed intermediate the substantially uniformly spaced-apart annular flanges positioned axially along the inner surface of each of the clamshells to retain the airway device in a preselected position in a patient’s airway. The inner surface of each grommet may be coated with an adhesive material, by way of example and not of limitation, a pressure sensitive adhesive (PSA) adapted to adhesively engage the outer surface of an airway device.

In still another embodiment, the airway stabilization system includes an adjustable ratchet mechanism adapted to releasably clamp ETT’s of different tube sizes.

In an embodiment, the carriage member includes a first release mechanism adapted to permit selective adjustment of the position of an airway device with respect to a patient’s vocal cords.

In still another embodiment, the airway stabilization system includes a second release mechanism adapted to selectively position the carriage member within the clamping member.

In yet another embodiment, the airway stabilization system includes a lateral position adjustment mechanism adapted to laterally adjust the position of the tower structure on the bridge or support member.

In an embodiment, the airway stabilization system includes a locking mechanism adapted to releasably lock the pivotally interconnected clamshells together circumferentially around the carriage member in stabilizing and supporting engagement therewith.

In another embodiment the airway stabilization system locking mechanism further includes a release mechanism, for example, a quick-release actuator or button whereby the c-shaped collars or clamshells may be easily and rapidly released from locking engagement with one another.

These and other features, aspects and advantages of the present invention will become apparent to those skilled in the art from the following detailed description of preferred embodiments taken in connection with the accompanying drawings, which are briefly summarized below, and by reference to the appended claims.

### BRIEF DESCRIPTION OF THE DRAWINGS

Referring now to the attached drawings which form a part of this original disclosure:

FIG. 1 is a front side perspective view of portions of an airway stabilization system including an airway device and a carriage member or collar positioned thereon in accordance with an embodiment of the present invention;

FIG. 2 is a front side perspective view of portions of a securing apparatus or stabilizer of the airway stabilization system of FIG. 1 including a supporting bridge or frame member, and tower structure or clamshell-type clamping member having an integrated carriage member or collar adjustably positioned in the tower structure and adapted to releasably engage an airway device;

FIG. 3 is a top plan view of the portions of the airway stabilization system of FIG. 2 showing the clamshell-type clamping member and the integrated carriage member or collar in an open position;

FIG. 4 is a front elevation view of portions of the airway stabilization system of FIGS. 2 and 3 as viewed from the top of a patient’s head;

FIG. 5 is a left side elevation view of portions of the airway stabilization system of FIGS. 2-4;

FIG. 6 is a bottom elevation view of the airway stabilization system of FIGS. 2-5 as viewed from a patient’s chin;

FIG. 7 is a top perspective view of the portions the securing apparatus or stabilizer of the airway stabilization system shown in FIG. 2;

FIG. 8 is a top plan view of the portions of the airway stabilization system of FIG. 3 showing the clamshell-type clamping member and the integrated carriage member or collar in a closed position;

FIG. 9 is a front elevation view of portions of the airway stabilization system of FIG. 8 as viewed from the top of a patient’s head;

FIG. 10 is a left side elevation view of portions of the airway stabilization system of FIGS. 8 and 9;

FIG. 11 is a bottom elevation view of the airway stabilization system of FIGS. 8-10 as viewed from a patient’s chin;

FIG. 12 is a top side perspective view of the portions the securing apparatus or stabilizer of the airway stabilization system shown in FIGS. 8-11;

FIG. 13 is a bottom side perspective view of the portions the securing apparatus or stabilizer of the airway stabilization system shown in FIG. 12;

FIG. 14 is a bottom elevation view of the airway stabilization system of FIGS. 6 and 7 showing the integrated carriage member or collar positioned in the tower structure or clamshell-type clamping member in a direction toward the patient which would position the attached tube deeper in the patient’s airway;

FIG. 15 is a top side perspective view of the airway stabilization system of FIG. 14;

FIG. 16 is a bottom side perspective view of the airway stabilization system of FIGS. 14 and 15;

FIG. 17 is a bottom elevation view of the airway stabilization system of FIG. 6 and 7 showing the integrated carriage member or collar positioned in the tower structure or clamshell-type clamping member in a direction away from a patient’s face which would position the attached tube more shallowly in the patient’s airway;

FIG. 18 is a top side perspective view of the airway stabilization system of FIG. 17;

FIG. 19 is a bottom side perspective view of the airway stabilization system of FIGS. 17 and 18;

FIG. 20 is a top plan view of an integrated carriage member or collar shown in an open position;

FIG. 21 is a top side perspective view of the integrated carriage member or collar of FIG. 20;

FIG. 22 is a top plan view of an integrated carriage member or collar shown in a closed position;

FIG. 23 is a top side perspective view of the integrated carriage member or collar of FIG. 22;

FIG. 24 is a bottom elevation view of the integrated carriage member or collar of FIGS. 20-23;

FIG. 25 is a front elevation view of the integrated carriage member of collar of FIGS. 20-24:

FIG. 26 is a front side perspective view of portions of a securing apparatus or stabilizer of an airway stabilization system including a supporting bridge or frame member, and tower structure or clamshell-type clamping member having an integrated carriage member or collar adjustably positioned in the tower structure and adapted to releasably engage an airway device in accordance with another embodiment of the present invention:

FIG. 27 is a side perspective view of a securing apparatus or stabilizer of the embodiment of FIG. 26 shown having an airway device positioned therein to illustrate the elements thereof more clearly;

FIG. 28.Ais a top view of an airway stabilization system illustrating the elements a carriage member or collar adjustment mechanism for securing airway devices of varying diameters shown fitted on a large diameter airway device;

FIG. 28.Bis a top view of an airway stabilization system illustrating the elements a carriage member adjustment mechanism for securing airway devices of varying diameters shown fitted on a small diameter airway device;

FIG. 29.Ais a top view of the airway stabilization system of FIGS. 26 and 28.A showing the elements of a mechanism for adjusting the insertion depth of an airway device having a large diameter in a patient;

FIG. 29.Bis a top view of the airway stabilization system device of FIGS. 26 and 28.Bshowing the elements of a mechanism for adjusting the insertion depth of an airway device having a small diameter in a patient;

FIG. 30 is a side perspective view of a securing apparatus or stabilizer of an airway stabilization system including a tower structure or clamshell-type clamping member having an individual grommet member adjustably positioned in the tower structure and adapted to releasably engage an airway device in accordance with an embodiment;

FIG. 31.Ais a side perspective view of a tower structure or clamshell-type clamping member shown in an open position and having multiple individual grommet members adjustably positioned in each of the c-shaped portions thereof and adapted to releasably engage an airway device having a small diameter in accordance with an embodiment;

FIG. 31.Bis a side perspective view of a tower structure or clamshell-type clamping member having individual grommet members adjustably positioned in each of the c-shaped portions thereof and adapted to releasably engage an airway device having a large diameter in accordance with an embodiment;

FIG. 32.Ais a side perspective view of a tower structure or clamshell-type clamping member illustrating the clamping member in a closed position encapsulating an individual grommet member, the clamping member being secured to the bridge or frame member by a release mechanism in accordance with an embodiment;

FIG. 32.Bis a side elevation view of an individual grommet member illustrating a radial snap or lock mechanism adapted to hold the two c-shaped halves thereof in a locked position in accordance with an embodiment;

FIG. 32.Cis a top plan view of the grommet member of FIG. 8.Bin accordance with an embodiment;

FIG. 33.Ais a partial sectional top perspective view of the airway stabilization system of FIG. 32.Ahaving portions of the bridge attachment and release mechanism removed to illustrate the elements thereof more clearly;

FIG. 33.Bis a partial sectional top perspective view of the airway stabilization system of FIG. 33.Ashowing the direction of lateral adjustment thereof on a track portion of the frame or bridge member;

FIG. 34.Ais a partial sectional top perspective view of an airway stabilization system having portions of the bridge attachment and release mechanism shown in an overlay configuration to illustrate the interaction thereof more clearly;

FIG. 34.Bis a bottom perspective view of the release button of the bridge attachment and release mechanism;

FIG. 35 is a side perspective view of the securing apparatus or stabilizer of FIG. 30 having an individual grommet installed therein showing the stabilizer in a closed position;

FIG. 36 is a side perspective view of the securing apparatus or stabilizer of FIG. 35 shown in an open position;

FIG. 37 Is a side perspective view of the securing apparatus or stabilizer of FIGS. 35 and 36 illustrating the individual grommet in a closed position;

FIG. 38.Ais a side elevation view of a pediatric airway stabilization system installed on a 7-year-old patient showing the frame or bridge positioning on the patient’s facial geometry;

FIG. 38.Bis a top elevation view of a pediatric airway stabilization system installed on a 7-year-old patient showing the frame or bridge positioning on the patient’s facial geometry;

FIG. 39.Ais a side elevation view of a pediatric airway stabilization system installed on a neonatal patient of less than three month’s age showing the frame or bridge positioning on the patient’s facial geometry;

FIG. 39.Bis a top elevation view of a pediatric airway stabilization system installed on a neonatal patient of less than three month’s age showing the frame or bridge positioning on the patient’s facial geometry;

FIG. 40 is a side perspective view of a pediatric airway stabilization system installed on an infant;

FIG. 41 is a top perspective view of a pediatric airway stabilization system installed on an infant;

FIG. 42 is a side perspective view of a pediatric airway stabilization system installed on an infant showing portions of the ETT positioned in the infant’s airway to better illustrate the positioning of an inflation balloon and depth indicator bands;

FIG. 43 is a top view of an airway stabilization system illustrating a supporting bridge or frame member, a tower structure or clamshell-type clamping member having an integrated carriage member or collar adjustably positioned in the tower structure and a carriage member or collar adjustment mechanism for securing airway devices of varying diameters shown in a closed and engaged position with respect to the tower structure in accordance with an embodiment;

FIG. 44 is a top view of the airway stabilization system of FIG. 43 illustrating the carriage member adjustment mechanism in a closed and disengaged or released position for adjusting the insertion depth of an airway device;

FIG. 45.Ais a top view of the airway stabilization system of FIG. 43 illustrating the airway device clamping interface force vector;

FIG. 45.Bis a top view of the airway stabilization system of FIG. 44 illustrating the carriage release force vectors;

FIG. 46.Ais a side perspective view of the airway stabilization system of FIGS. 19-21 illustrating a snap locking mechanism in accordance with an embodiment; and

FIG. 46.Bis a side perspective view of the airway stabilization system of FIGS. 43-45 illustrating a zip-tie locking mechanism in accordance with an embodiment.

### DETAILED DESCRIPTION OF THE PREFERRED EMBODIMENTS

Selected embodiments of the present invention will now be explained with reference to the drawings. It will be apparent to those skilled in the art from this disclosure that the following descriptions of the embodiments of the present invention are provided for illustration only and not for the purpose of limiting the invention as defined by the appended claims and their equivalents.

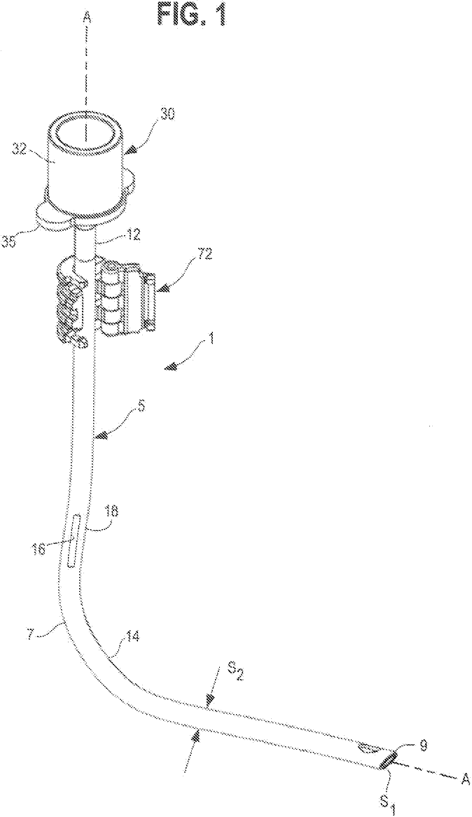

Referring initially to FIG. 1, portions of an airway stabilization system shown generally at numeral 1 are illustrated in accordance with an embodiment of the present invention. The airway stabilization system is used to maintain an airway in a human (or animal patient in veterinary applications) under conditions where natural respiration is impossible or severely compromised. The airway stabilization system includes an airway device depicted generally at 5 which has a flexible elongate body 7 extending along an axis A-A and having a length, an internal diameter S₁ and an external diameter S₂, a proximal (patient-end) portion 9, a distal (machine end) portion 12 and a continuous sidewall 14 having an internal surface 16 and an external surface 18 extending between the proximal and the distal ends. A connector assembly 30 is operatively connected to the machine end 12 of the elongate body of the airway device and is adapted to connect a respiratory connector, also referred to as a 15 mm connector, 32 to the airway device. The connector also includes “wings” 35 that are grips for inserting / removing the connector into / from the endotracheal tube.

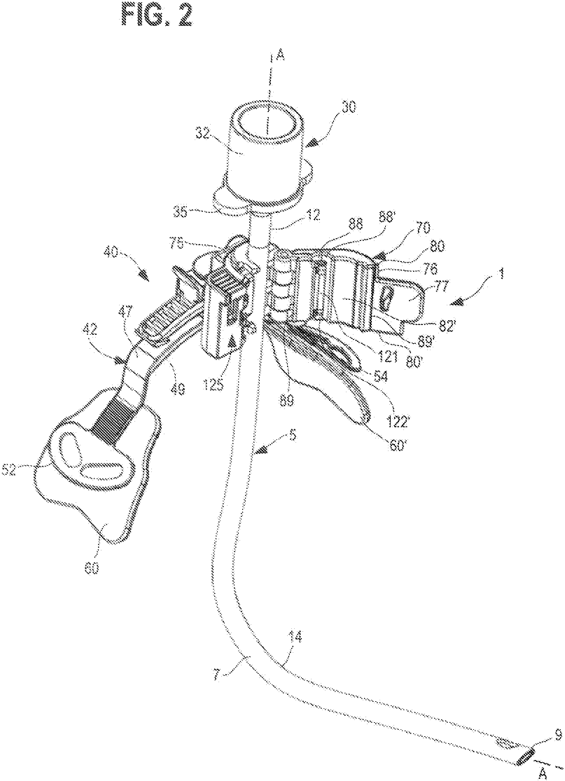

The airway stabilization system also includes a securing apparatus 40 (FIG. 2), portions of which are shown at 72 in FIG. 1, the airway device and securing apparatus cooperating to maintain an air passageway to a patient’s lungs via the patient’s mouth, oral cavity, throat, past a patient’s vocal cords or larynx into a patient’s trachea and to a patient’s carina (the point where the trachea bifurcates into the bronchial tubes) for respiration of the patient. By way of example and not of limitation, the airway device may be in the form of an endotracheal tube (ETT) as shown in the accompanying figures, one of several commercially available endotracheal tubes or one of several commercially available supraglottic airway devices such as a King LT™ airway device manufactured by King Systems, Noblesville, Indiana or a laryngeal mask airway (LMA) such as a LMA Classic™ manufactured by LMA North America, San Diego, California.

FIG. 2 depicts the details of the securing apparatus 40 positioned on an airway device 5 and illustrated in an open configuration in which it may be selectively moved coaxially along axis A-A either proximally or distally depending upon a patient’s facial geometry and anatomical structure to ensure proper insertion depth and maintenance of the airway device 5 in the patient’s trachea. As the terms are used herein, proximal or proximally refer to a direction or position toward the patient, and distal or distally refers to a position or location away from the patient. When referring to movement in relation to the tube itself, references will be to the machine end or patient end.

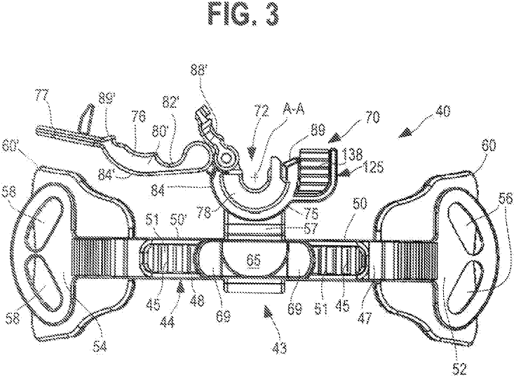

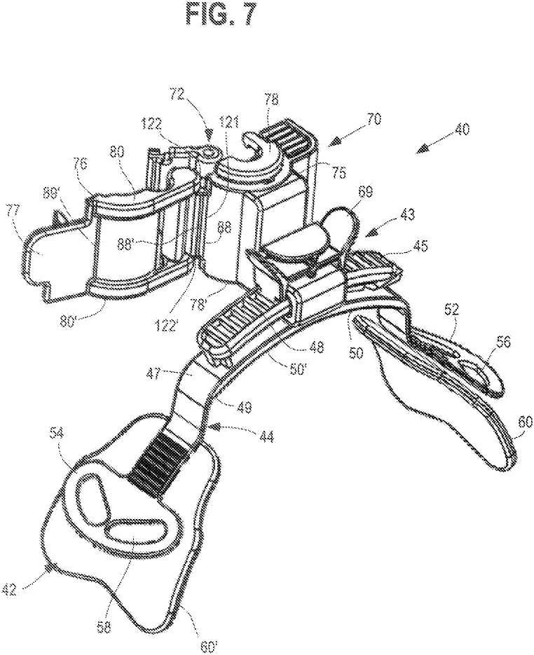

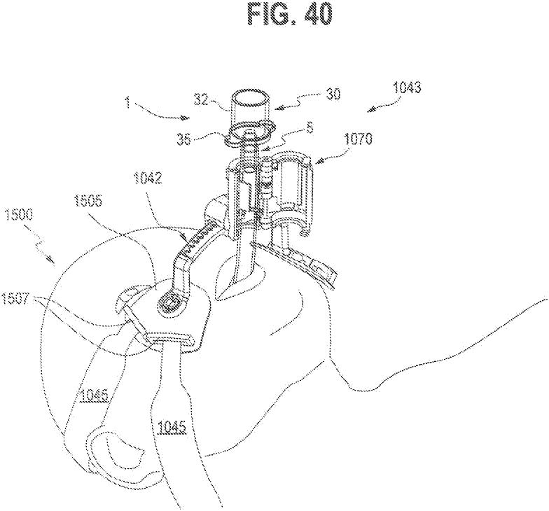

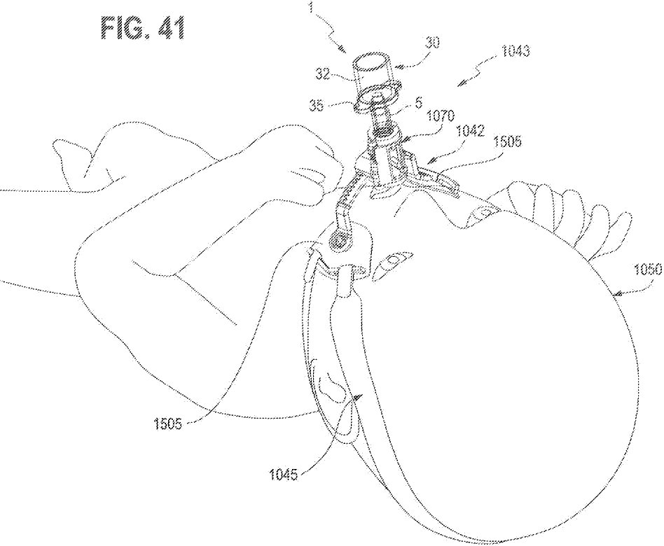

As shown in FIG. 2 and in greater detail in FIGS. 3 and 7, the securing apparatus 40 includes a supporting bridge or frame 42 which may be secured to a patient’s face by a suitable attachment apparatus, by way of example and not of limitation, one or more straps 1045 (FIGS. 40-42) extending around the patient’s head and securable by buckles. Velcro or other suitable attachments, as is known in the art. The bridge or frame is preferably of unitary construction and in a generally symmetrical configuration contoured to permit it to conform to a patient’s face when it is secured in position. It may be formed of plastic, rubber, metal, composite material or other suitable materials having the desired physical properties for the application. The frame includes a body 44, an upper or outer surface 47 facing away from the patient and a lower or bottom surface 49 facing toward the patient, the upper and lower surfaces being interconnected by a pair of oppositely disposed, spaced apart side portions 50, 50′ and first and second oppositely disposed end portions 52 and 54. As best seen in FIG. 3, each end portion has at least one aperture or slot 56 and 58 formed therein respectively and adapted to receive the attachment apparatus or strap 1045. The frame is generally symmetric about a patient’s oral cavity and nose, thereby facilitating ease of positioning thereof on a patient’s face and may also include a cheek pad or layer 60, 60′ of cushioning material releasably secured to a lower surface 53, 53′ of each respective end to reduce pressure on a patient’s face and to provide additional comfort for the patient. By way of example and not of limitation, the cushioning material may be a hydrocolloid absorbent, waterproof material, or a silicone gel pad for use when a patient having burns, skin tears, rashes, facial hair where an adhesive would be contraindicated. The cushioning material is operatively secured to ends 52 and 54 by a suitable securing material such as a releasable adhesive or bonding agent or a thin Velcro® brand fastener to allow changeout or replacement thereof when the cheek pads become soiled without being bulky or uncomfortable. In an embodiment, a skin contact adhesive is applied to the cushioning material that will have a perforated pattern to allow moisture to wick to the underlying absorbent hydrocolloid material, thus permitting multi-day wear of the airway stabilization system.

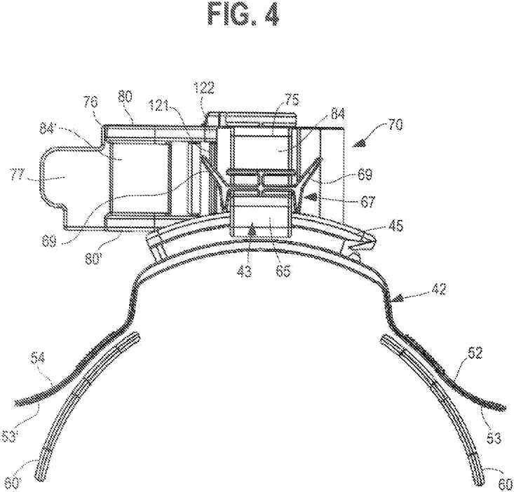

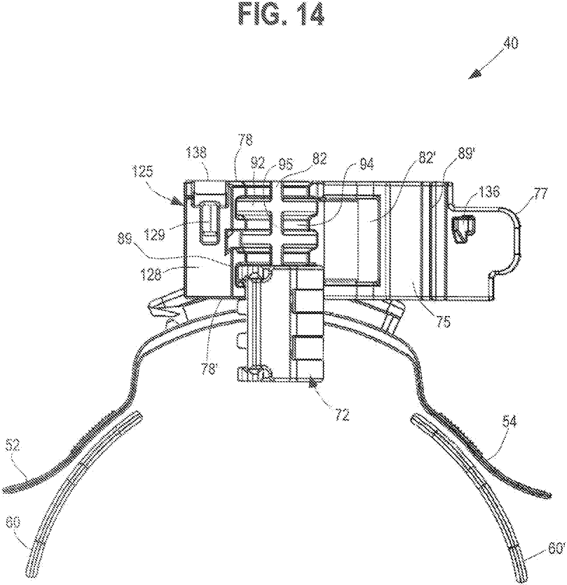

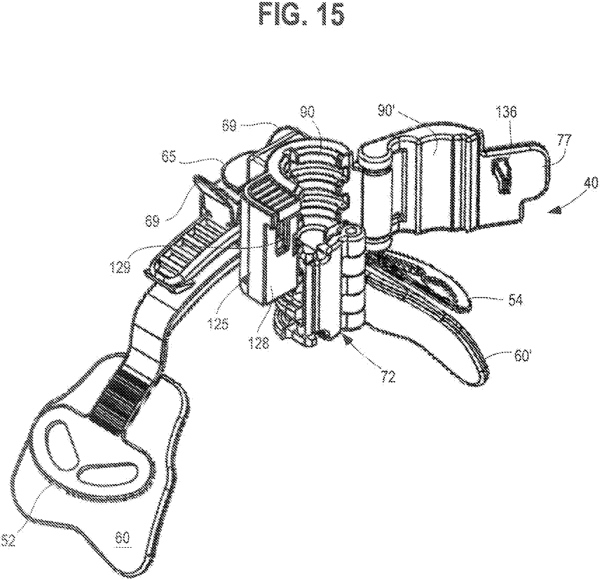

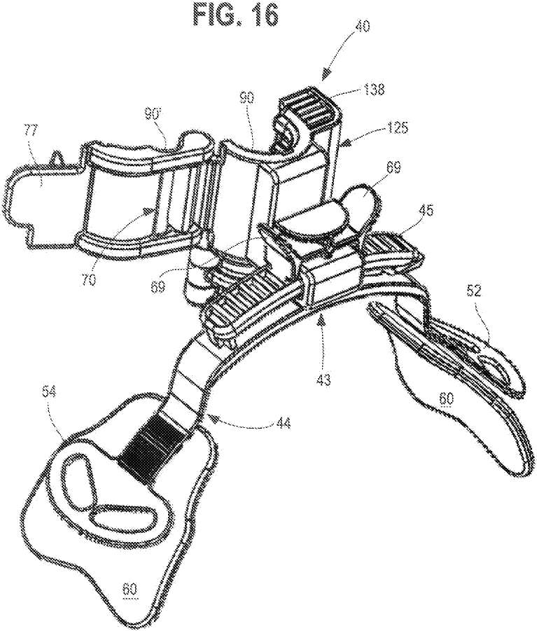

Referring again to FIGS. 2, 3, and 7 and also to FIGS. 4-6, the securing apparatus 40 includes a generally cylindrically shaped tower structure or clamshell-type clamping member 70 operatively connected to the frame 42 via an attachment device 43, as described in greater detail below. The clamping member is configured to interact in clamping engagement with the continuous sidewall of the airway device via a carriage member or collar 72 adjustably positioned in the tower structure to prevent clinically significant movement of the distal (patient-end) of the airway device with respect to the vocal cords of the patient. The collar extends in a substantially perpendicular direction from the outer surface 47 of the frame 42 coaxially along axis A-A. The clamping member 70 includes a pair of oppositely disposed pivotally interconnected c-shaped collars or clamshells 75, 76, each collar or clamshell having a length, first end and second ends 78, 78' and 80, 80′, an inner surface 82, 82′ (FIG. 14) and an outer surface 84, 84′. Each of the clamshells has a pair of generally parallel extending edge surfaces 88, 88′ and 89, 89′ respectively, the edge surfaces and the corresponding c-shaped clamshell each defining an opposed, semi-cylindrically shaped cavity 90, 90′ about the axis A-A. These cavities are most clearly shown in FIGS. 15 and 16.

Referring to FIG. 14, in an embodiment the inner surface 82 of clamping member or collar 75 includes a plurality of substantially uniformly spaced-apart annular flanges 92 positioned axially along the inner surface thereof and extending substantially radially inwardly therefrom, and a plurality of structural recesses 94 positioned axially along the inner surface of the collar or clamshell intermediate an adjacent two of the plurality of substantially uniformly spaced-apart annular flanges. At least one structural recess 95 extends radially inwardly from inner surface 82 intermediate first and second ends 78, 78' in a direction parallel to axis A-A. As will be described below, the recess 95 interacts in mating engagement and cooperates with a corresponding at least one rib 119 formed in carriage member or collar 72 to prevent rotation and maintain the alignment of the carriage member or collar 72 in the tower structure.

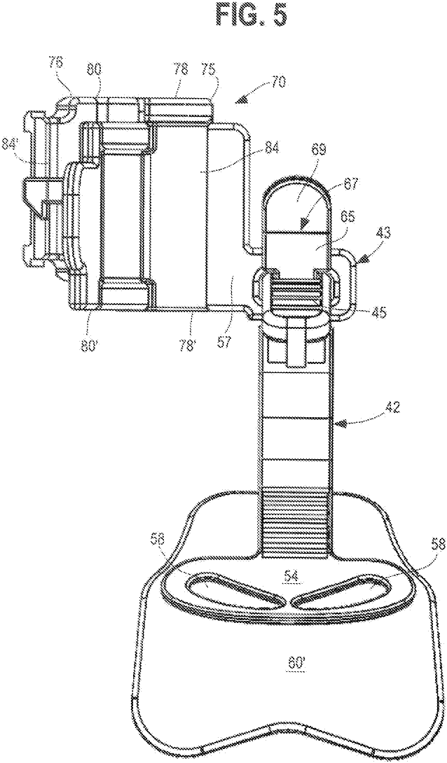

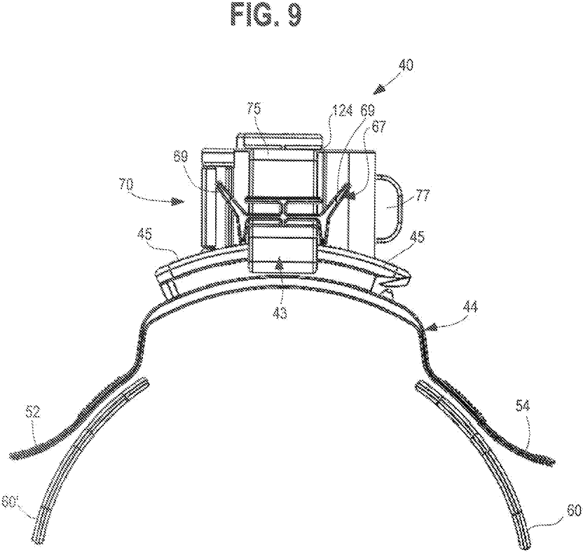

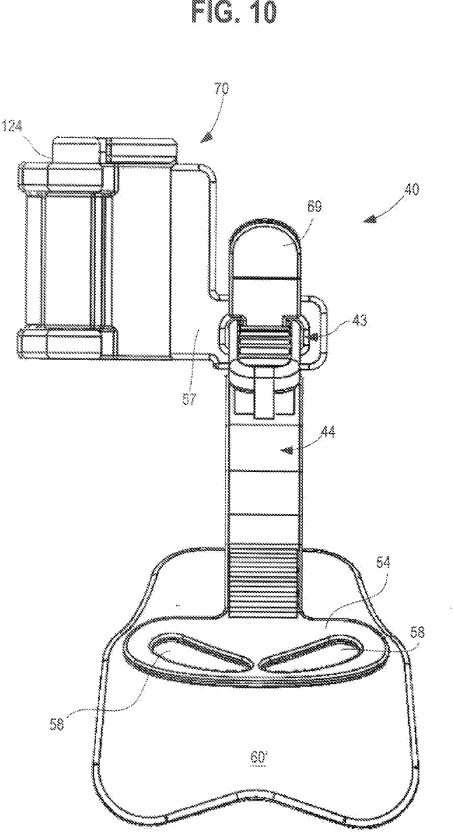

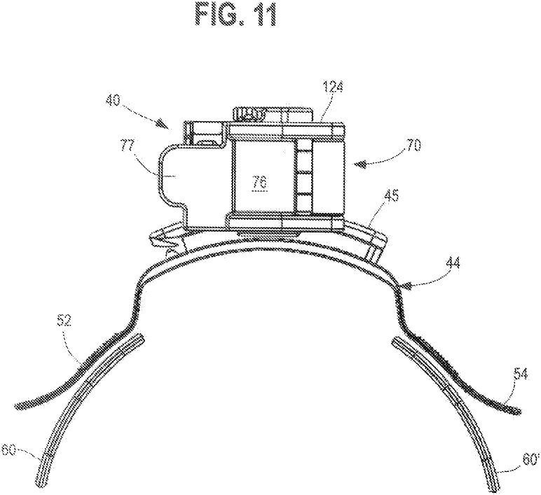

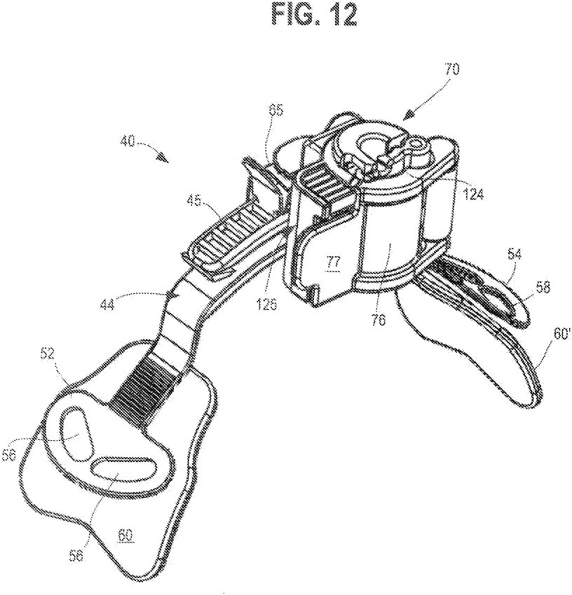

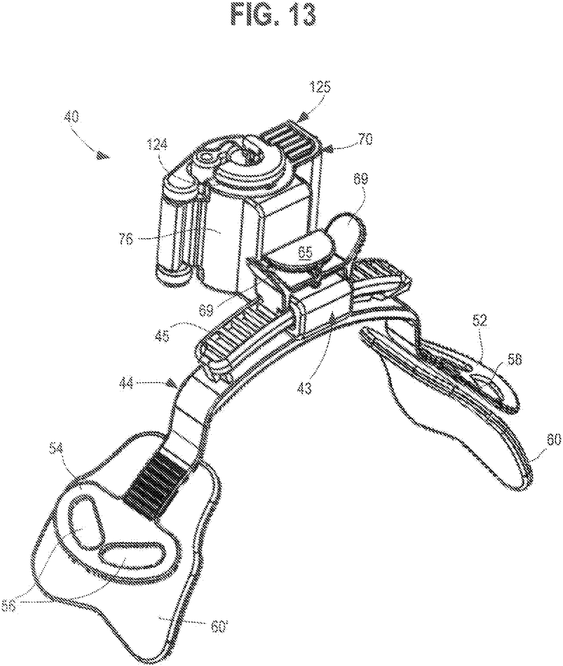

As shown in FIGS. 2-3, in operation the collars or clamshells 75 and 76 are pivotally interconnected, for example, by a pin 121 which extends through a plurality of hinge members 122, 122′ operatively connected to edges 88 and 88′ of the clamshells 75 and 76, respectively. A handle or operating arm 77 extends radially outwardly from edge 89′ of collar 76 and is adapted to facilitate opening and closing of the clamshells. Referring to FIGS. 5 and 9-13, the clamshells are shown in a closed position and in mating contact with one another along edges 88, 88′ and 89, 89′, thereby forming a cavity 124 defined by the opposed semi-cylindrically shaped cavities 90 and 90′. Referring to FIG. 14, the collars are retained in locking engagement by a releasable latch mechanism 125. The latch mechanism includes a housing 128 having an aperture 129 formed therein, the aperture being adapted to receive a latch member or finger 136 operatively connected to the operating arm 77 and extending outwardly therefrom. A release button 138 is urged into locking engagement by a spring or other biasing mechanism with the finger 136 when the collars or clamshells 75 and 76 are closed about the carriage member 72. Depressing the button 138 releases the finger which, in turn permits the clamshell or collar 76 to be rotatably opened, thus releasing the carriage member for either positional adjustment along the axis A-A or removal from the tower structure 70.

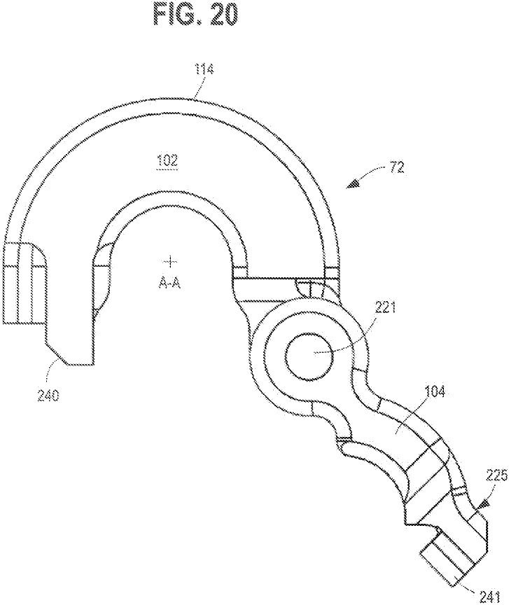

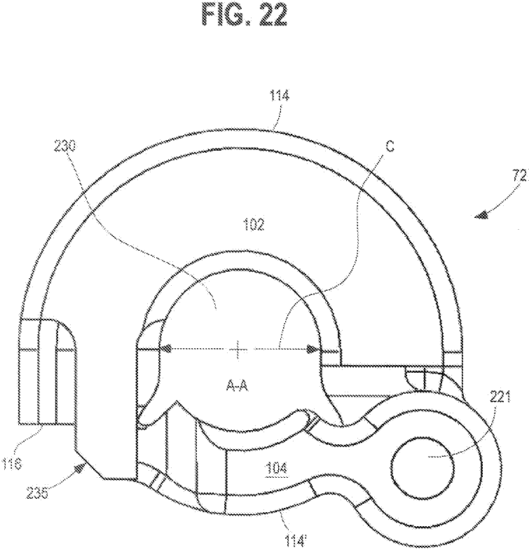

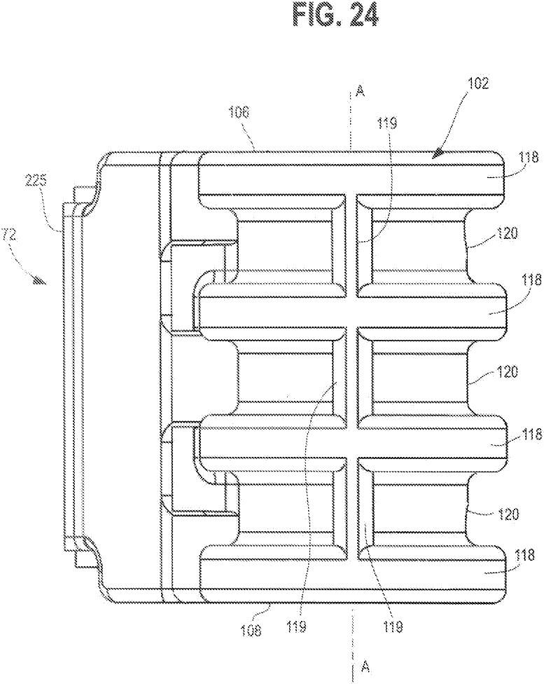

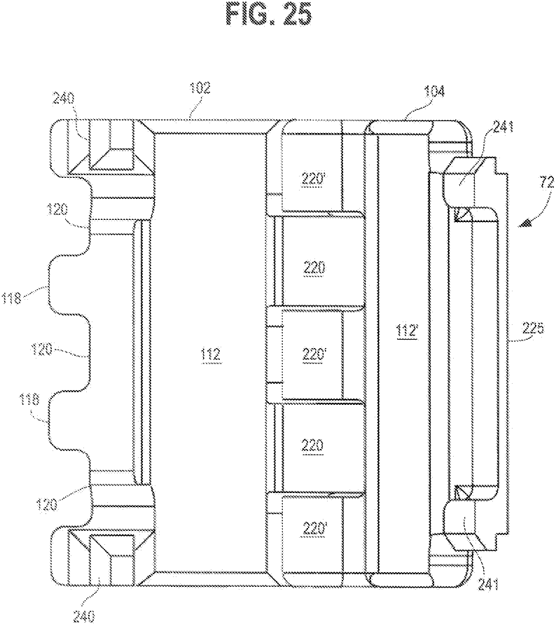

The elements of the carriage member or collar 72 are illustrated FIGS. 20-25. The carriage member is formed by a pivotally interconnected elongate c-shaped cylindrical member 102 and a curvilinear cover or closure member 104, each of which includes first and second ends 106, 108 and 106′, 108′ respectively and a body portion 110, 110′, each body portion having a curved, semi-cylindrical inner surface 112,112', an outer surface 114, 114′, and a pair of generally parallel extending edge surfaces 115, 115′ and 116, 116′ respectively’ extending therebetween. The outer surface of cylindrical member 102 includes at least one annular flange 118 and structural recess 120 extending radially outwardly from the outer surface 114 and adapted to operatively interact with one of the plurality of structural recesses formed intermediate an adjacent two of the plurality of substantially uniformly spaced-apart annular flanges positioned axially along the inner surface of clamshell 75. As best shown in FIG. 24, at least one rib or flange 119 extends radially outwardly from outer surface 114 intermediate first and second ends 106, 108 in a direction parallel to axis A-A. The at least one rib 119 interacts in mating engagement and cooperates with the at least one structural recess 95 formed in clamshell 75 to maintain alignment therebetween. While in the embodiment shown only the outer surface 114 of cylindrical member 102 includes at least one annular flange and structural recess, closure member 104 may also be so structured without departing from the scope of the present invention.

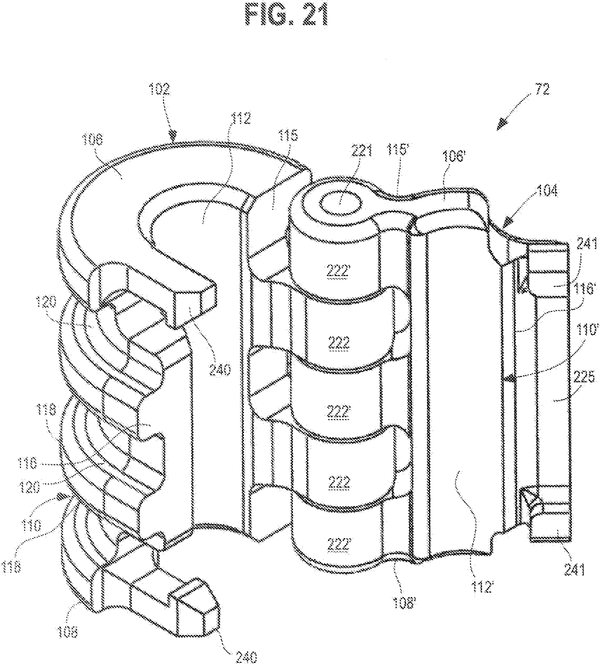

Referring now to FIGS. 20 and 21, the elements of the interconnection of the elongate c-shaped cylindrical member 102 and the curvilinear cover or closure member 104 are shown. The members are pivotally interconnected, for example, by a pin 221 which extends through a plurality of hinge members 222, 222′ operatively connected to edges 115 and 115′ of the carriage or collar members 102 and 104, respectively. A handle or operating arm 225 extends radially outwardly from edge 116′ of closure member 104 and is adapted to facilitate opening and closing of the collars.

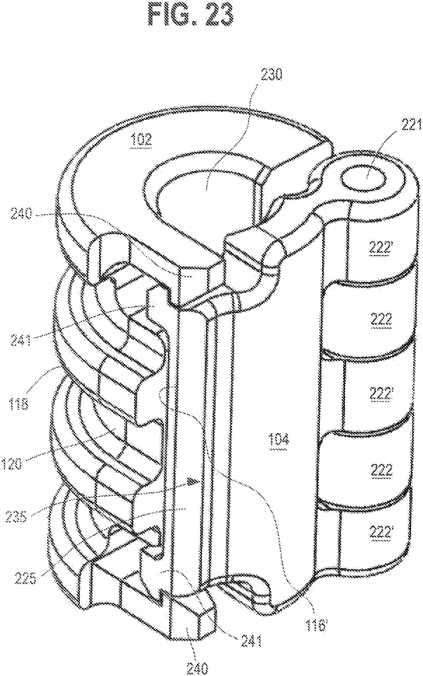

The carriage members are shown in a closed position and in mating contact with one another along edges 116 and 116′ in FIGS. 21, 22 and 23, thereby forming a cavity 230 having a radius C. defined by the opposed semi-cylindrically shaped inner surfaces 112, 112′. The collars are retained in locking engagement about an airway device by a latch mechanism shown as element 235 in FIG. 23. The latch mechanism includes a pair of juxtaposed latch members or fingers 240, 241 which snap into locking engagement with one another when the cover or closure member 104 is urged into locking engagement with the c-shaped cylindrical member 102. In addition, the inner surfaces 112 and 112′ of the cylindrical and clamping members may be coated with an adhesive material, by way of example and not of limitation, a pressure sensitive adhesive (PSA) adapted to adhesively engage the outer surface of an airway device. In an embodiment, a bonding agent may be applied or mechanically injected into an interface intermediate the outer surface of the airway device and one or both of the inner surfaces 112, 112′ to permanently affix the collar to the airway device. In yet another embodiment, the inner surfaces may be textured, for example, like the surface texturing found on a porcupine quill, to selectively prevent axial motion along the A-A axis of an airway device in one or both axial directions.

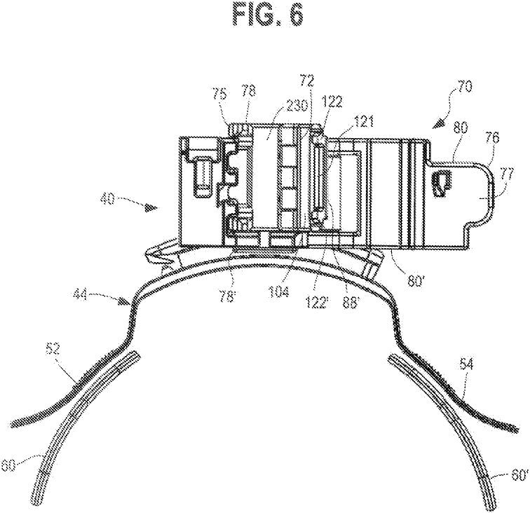

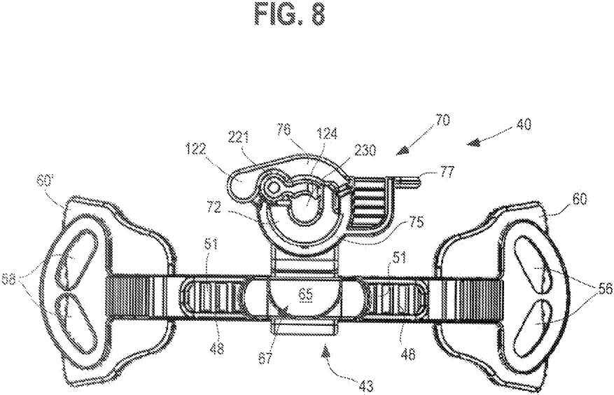

The interactive and cooperative locking engagement between the tower structure or clamshell-type clamping member 70 and the carriage member or collar 72 provides selective coaxial positioning of an airway device along axis A-A either proximally (deeper into the patients airway) or distally (more shallowly relative to the patients airway) depending upon a patient’s facial geometry and anatomical structure to ensure proper insertion depth and maintenance of the airway device in the patient’s trachea. For example, the securing apparatus 40 is illustrated in an open configuration in FIGS. 6 and 7 with the carriage member or collar 72 centrally positioned within the tower structure 70. Once secured on a patient with an airway device positioned in cavity 230, if no additional adjustments to the insertion depth of the airway device are required, the clinician will close the curvilinear cover or closure member 104 of the carriage member into secure engagement with the airway device and then close the collars or clamshells 75 and 76 into locking engagement with one another and with the carriage member. Although the collar closure member and clamshell (tower) may be closed separately, the system is configured so that the closure of both members may be performed with a single maneuver. The airway stabilization system with the securing apparatus in a closed configuration having the carriage member centrally located within the tower structure is shown in FIGS. 6-13.

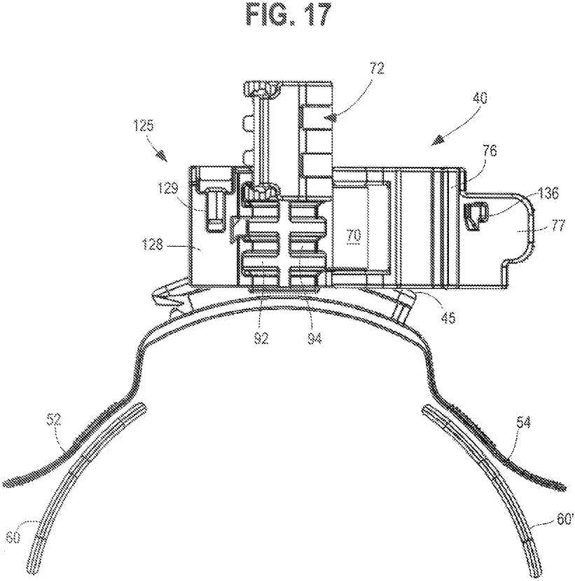

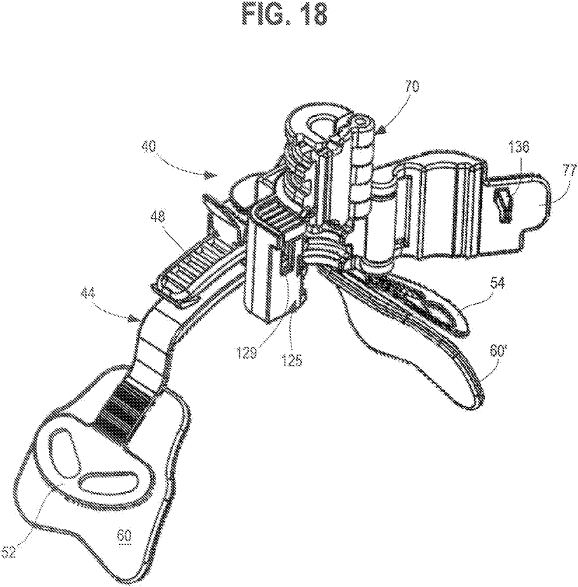

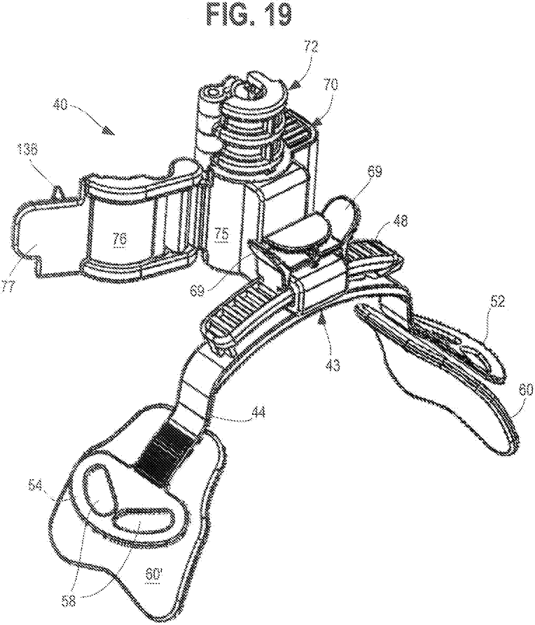

Should position adjustments of an airway device be required axially in response to a patient’s anatomical structure to establish and maintain proper insertion depth, the carriage member or collar 72 with the tube attached may be moved in either a direction to position the tube deeper in the patient’s trachea as shown in FIGS. 14-16 or in a direction to position the tube more shallowly in the patient’s trachea as shown in FIGS. 17-19. In either scenario, once proper airway insertion depth established, the curvilinear cover or closure member 104 of the carriage member and the collars or clamshells 75 and 76 will be closed and locked as described above, thereby preventing unintended movement of the airway device and/or extubation of the patient in response to the application of forces thereto in any direction.

In addition to the axial position adjustability provided by the airway stabilization system of the instant invention as hereinabove described, the lateral position of an airway device with respect to a patient’s oral cavity may also be selectively adjusted in response to the patient’s facial geometry and anatomical structure. Referring again to FIGS. 3-5, the securing apparatus 70 is operatively connected to the bridge or frame 42 by a connecting member or beam 57 portion of the attachment device 42. The structure of the bridge or frame 42 and the attachment device or apparatus 43 enables precise alignment of an airway device with the patient’s mouth or oral cavity and clearances between the apparatus and the patient’s face. The arrangement of the interacting components of the system permits its use over a broad range of facial geometries, adapted to be used with small or large diameter airway devices as the situation dictates.

As shown more clearly in FIGS. 3, 7 and 8, a linear track 45 is operatively connected to upper surface 47 of the supporting bridge or frame 42 and includes a plurality of spaced apart teeth 48 extending intermediate the side portions 50 and 50′ in a direction perpendicular to the axis A-A. Each of the plurality of teeth is separated from an adjacent one of the plurality of teeth by a space or recess 51, the plurality of teeth and recesses being adapted to releasably engage a securing or gripping mechanism 65 forming part of the attachment device 43. The securing mechanism includes a pinch release mechanism 67 having a pair of juxtaposed ears or levers 69, each ear or lever including a distal end 71 positioned in one of the plurality of recesses 51 and adapted to releasably engage an adjacent one of the plurality of teeth 48. When the ears are moved toward one another, for example by being manually compressed or squeezed, each of the distal ends moves out of its position in a respective one of the plurality of recesses 51, thus permitting lateral adjustment of the position of the securing apparatus 70. A biasing mechanism (not shown), for example, a compression spring positioned between the juxtaposed ear or levers is adapted to maintain the distal ends thereof in releasable engagement with the plurality of teeth and spaces or recesses on the supporting bridge or frame.

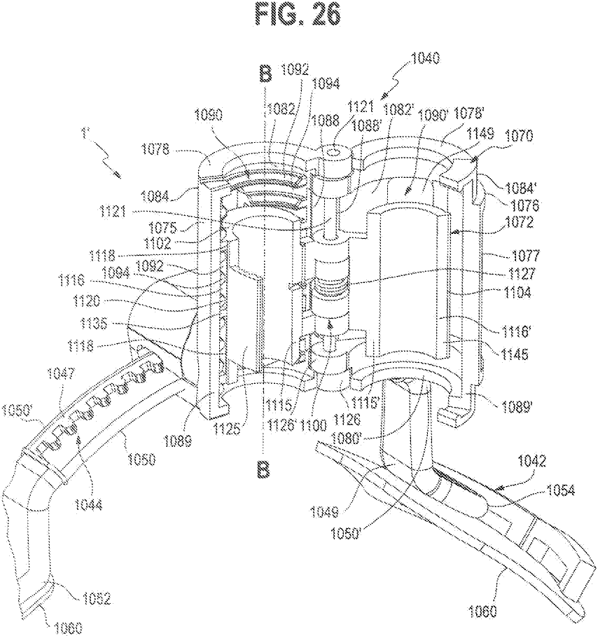

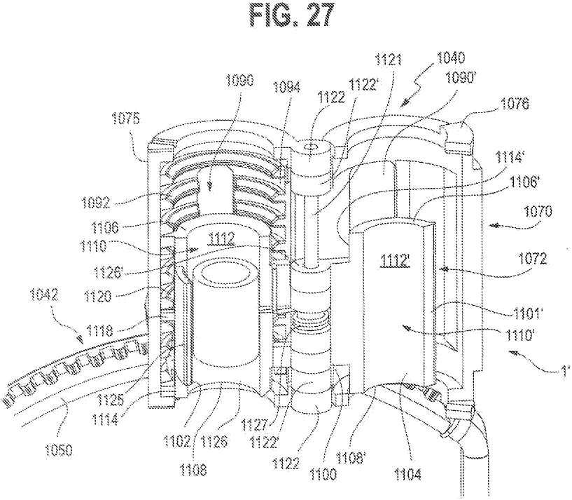

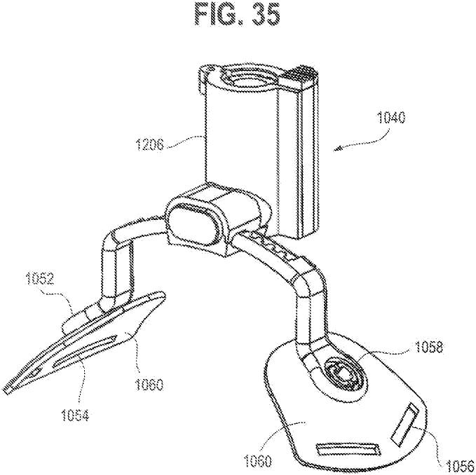

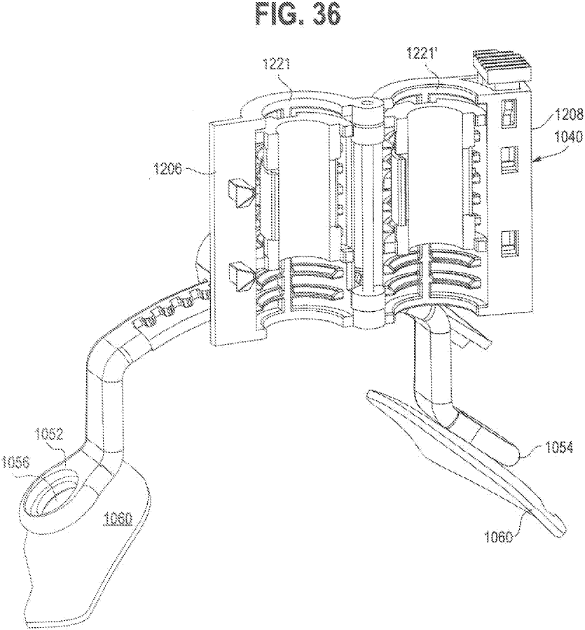

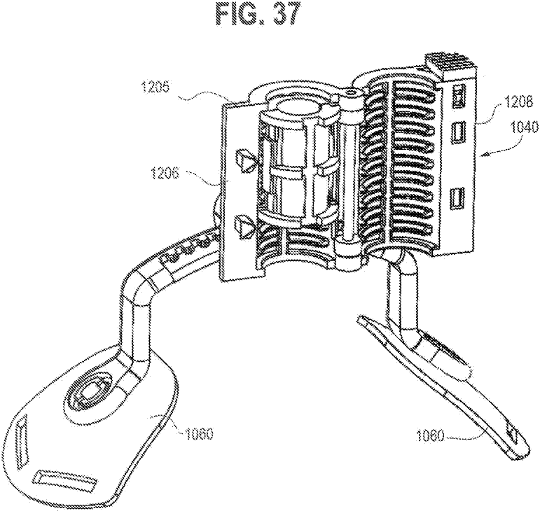

Referring now to FIGS. 26 and 27, portions of a securing apparatus 1040 are depicted in greater detail in accordance with another embodiment of the present invention. The securing apparatus 1040 includes a supporting bridge or frame 1042 which may be secured to the patient’s face by a suitable attachment apparatus, by way of example and not of limitation, one or more straps 1045 (FIGS. 40-42) extending around the patient’s head and securable by buckles, Velcro or other suitable attachments, as is known in the art. The bridge or frame is preferably of unitary construction and in a generally symmetrical configuration contoured to permit it to conform to a patient’s face when it is secured in position. It may be formed of plastic, rubber, metal, composite material, or other suitable materials having the desired physical properties for the application. The frame includes a body 1044, an upper or outer surface 1047 facing away from the patient and a lower or bottom surface 1049 facing toward the patient, the upper and lower surfaces being interconnected by a pair of oppositely disposed, spaced apart side portions 1050, 1050′ and first and second oppositely disposed end portions 1052 and 1054. As best seen in FIGS. 35 and 36, the frame is generally symmetric about a patient’s oral cavity and nose, thereby facilitating ease of positioning thereof on a patient’s face and may also include a cushioning material or layer 1060 secured to the lower surface of each end to provide additional comfort for the patient. Each layer 1060 has at least one an aperture or slot 1054 and 1056 formed therein respectively and adapted to receive the attachment apparatus or strap 1045.

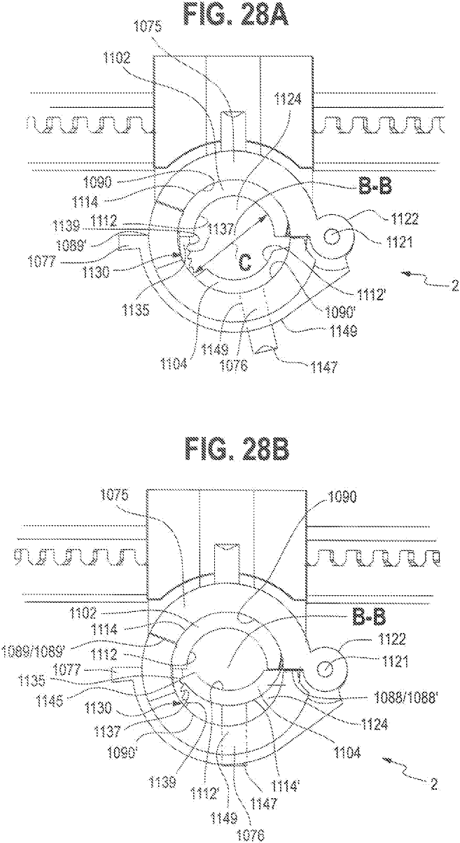

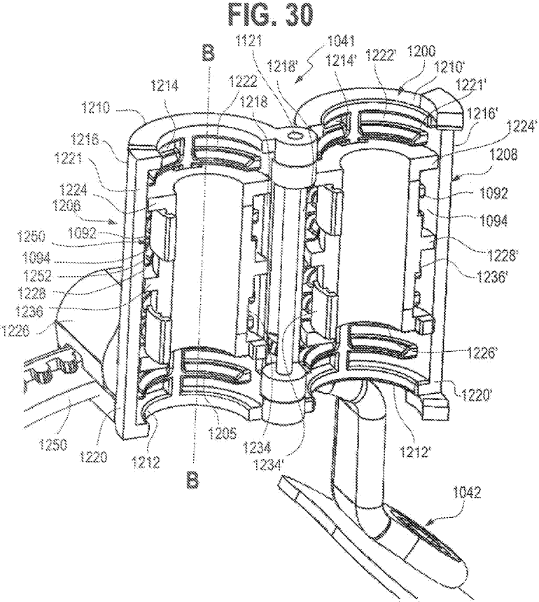

Referring again to FIGS. 26 and 27, the securing apparatus 1040 includes a generally cylindrically shaped tower structure or clamshell-type clamping member 1070 operatively connected to the frame 1042, as described in greater detail below. The clamping member is configured to interact in clamping engagement with the continuous sidewall of the airway device via a carriage member or collar 1072 adjustably positioned in the tower structure to prevent clinically significant movement of the patient end of the airway device with respect to the vocal cords of the patient. The collar extends in a substantially perpendicular direction from the outer surface 1047 of the frame 1042 along axis B-B. The clamping member includes a pair of oppositely disposed pivotally interconnected c-shaped collars or clamshells 1075, 1076, each collar or clamshell having a length, first end and second ends 1078, 1078' and 1080, 1080′, an inner surface 1082, 1082′ and an outer surface 1084, 1084′. Each of the clamshells has a pair of generally parallel extending edge surfaces 1088, 1088′ and 1089, 1089′ respectively, the edge surfaces and the corresponding c-shaped clamshell each defining an opposed, semi-cylindrically shaped cavity 1090, 1090′ about the axis B-B. These cavities are most clearly shown in FIG. 27 from the side and in FIGS. 28.A and 28.B from the top. In the embodiment of FIG. 26, the inner surface 1082 of clamping member or collar 1075 includes a plurality of substantially uniformly spaced-apart annular flanges 1092 positioned axially along the inner surface thereof and extending substantially radially inwardly therefrom, and a plurality of structural recesses 1094 positioned axially along the inner surface of the collar or clamshell intermediate an adjacent two of the plurality of substantially uniformly spaced-apart annular flanges. As will be described in greater detail below, in the embodiment of FIG. 30, both clamshells, 1206 and 1208 include the inwardly extending annular flanges and structural recesses 1092, 1094 positioned axially along the respective inner surfaces thereof.

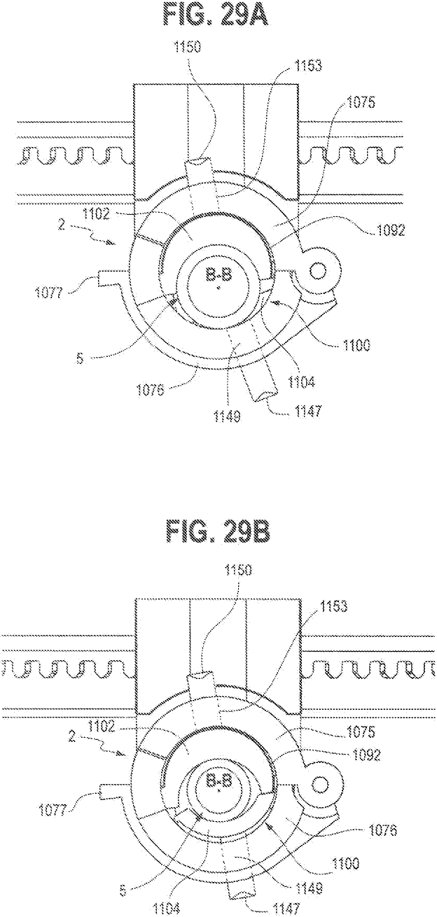

The airway stabilization system 1 of the embodiment of FIGS. 26, 27 and airway stabilization system 2 of the embodiment of FIGS. 28 and 29 further include an integrated carriage member or collar 1100. The carriage member is formed by a pair of pivotally interconnected elongate c-shaped cylindrical members 1102, 1104, each positioned within and operatively connected to a respective one of the c-shaped collars or clamshells 1075, 1076 and extending outwardly from the patient’s face coaxially with the longitudinal axis B-B of the clamping member. Each of the elongate cylindrical members includes first and second ends 1106, 1108 and 1106′, 1108′ respectively and a body portion 1110, 1110′ having an inner surface 1112, 1112′, an outer surface 1114, 1114′, and a pair of generally parallel extending edge surfaces 1115, 1115′ and 1116, 1116′ respectively’ extending therebetween. The outer surface of cylindrical member 1102 includes at least one annular flange 1118 and structural recess 1120 extending radially outwardly from the outer surface 1114 and adapted to operatively interact with one of the plurality of structural recesses formed intermediate the substantially uniformly spaced-apart annular flanges positioned axially along the inner surface of clamshell 1075. While in the embodiment shown only the outer surface 1114 of cylindrical member 1102 includes at least one annular flange and structural recess, cylindrical member 1104 may also be so structured without departing from the scope of the present invention. In addition, the inner surfaces of one or both of the cylindrical members of the carriage member or collar 1072 may be coated with an adhesive material, by way of example and not of limitation, a pressure sensitive adhesive (PSA) adapted to adhesively engage the outer surface of an airway device. In an embodiment, a bonding agent may be applied or mechanically injected into an interface intermediate the outer surface of the airway device and one or both of the inner surfaces to permanently affix the collar to the airway device. In yet another embodiment, the inner surfaces may be textured, for example, like the surface texturing found on a porcupine quill, to selectively prevent axial motion along the B-B axis of an airway device in one or both axial directions.

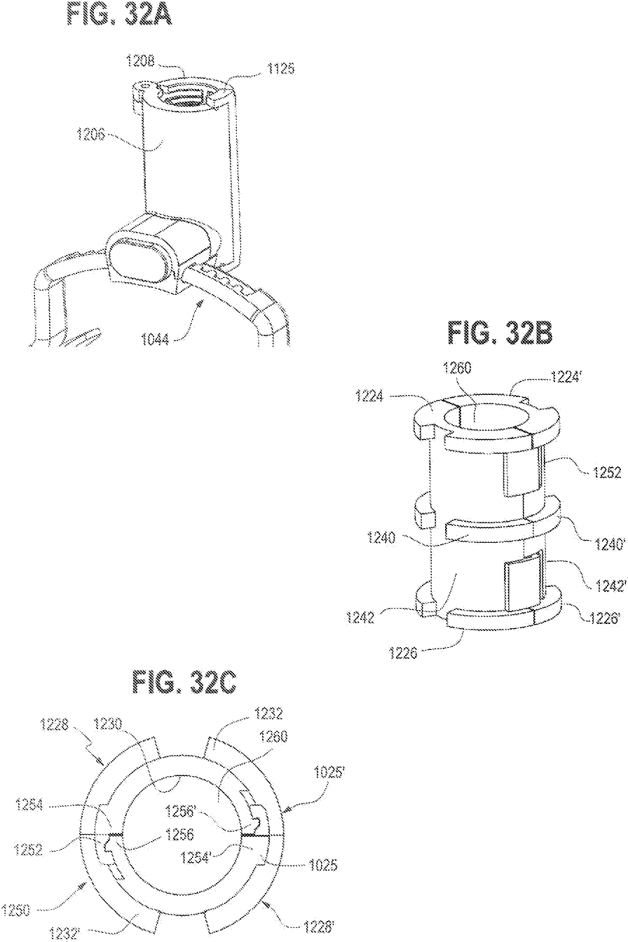

In operation the c-shaped collars or clamshells 1075 and 1076 are pivotally interconnected, for example, by a pin 1121 which extends through a plurality of hinge members 1122, 1122′ operatively connected to edges 1088 and 1088′ of the clamshells 1075 and 1076, respectively. A handle or operating arm 1077 extends radially outwardly from edge 1089′ of collar 1078′ and is adapted to facilitate opening and closing of the clamshells. Referring to FIGS. 28.A, 28.B, the clamshells are shown in a closed position and in mating contact with one another along edges 1088, 1088′ and 1089, 1089′, thereby forming a cavity 1124 defined by the opposed semi-cylindrically shaped cavities 1090 and 1090′. The collars are retained in locking engagement by a releasable latch mechanism shown as element 1125 in FIGS. 28.A, 28.Band also in FIG. 32.A.

The cavity has an inner diameter C and is adapted to releasably engage and enclose the pivotally interconnected elongate c-shaped cylindrical members 1102, 1104 of integrated carriage member or collar 1100. The c-shaped cylindrical members 1102 and 1104 are also pivotally interconnected by the pin 1121 which extends through a plurality of hinge members 1126, 1126′ operatively connected to edges 1115 and 1115′ of the c-shaped collars of the carriage member. A rotational force which urges the c-shaped carriage members 1102, 1104 into releasable engagement with the clamshells 1075, 1076 respectively is provided by a spring member, for example a coil spring 1127, positioned on the pin 1121 intermediate hinge members 1126, 1126′. More specifically, the spring-provided rotational force holds the at least one annular flange 1118 and structural recess 1120 extending radially outwardly from the outer surface 1114 of cylindrical member 1102 in operative engagement with one of the plurality of structural recesses 1094 formed intermediate the substantially uniformly spaced-apart annular flanges 1092 positioned axially along the inner surface of clamshell 1075. This novel feature permits rapid engagement and disengagement of the tower clamshells and the carriage members or collars whereby the position of an airway device in a patient’s trachea may be quickly and accurately adjusted in response to a patient’s anatomy and situational events arising during treatment requiring rapid response. The c-shaped collars are adjustably latched together in response to the varying sizes of airway devices used to intubate the patient by a latch or other suitable interlocking feature 1130. In the embodiment shown, the interlocking feature is in the form of a ratchet having a curvilinear member 1135 operatively connected to edge 1116. Member 1135 includes a plurality of ratchet teeth 1137 formed integrally on or operatively secured to an inner surface 1139 thereof, the ratchet teeth being structured and arranged to selectively engage a rib or flange 1145 extending along and radially outwardly from edge or surface 1089′. By way of example and not of limitation. FIG. 28.A illustrates the stabilization system releasably clamped circumferentially around an airway device having a 6 mm outer diameter elongate body 7 in stabilizing and supporting engagement therewith. The embodiment of FIG. 28.B depicts the stabilization system clamped circumferentially around an airway device having a smaller 4.5 mm body 7 in stabilizing and supporting engagement therewith. The engaged positions of the pivotally interconnected elongate c-shaped cylindrical members 1102, 1104 relative to one another may be selectively adjusted by either pushing on a control or release button 1147 operatively connected to elongate, c-shaped cylindrical member 1104. The release button extends through an aperture 1149 formed in the clamshell 1076 and is adapted to rotatably move the cylindrical members closer together. Conversely, by lifting the curvilinear member 1135 to release the rib 1145 from operative engagement with the ratchet teeth 1137, the cylindrical members may be moved farther apart to accommodate an airway device having a larger diameter.

Referring now to FIGS. 29.A and 29.B, the integrated carriage device 1100 is shown in a released position in which it is disengaged from the uniformly spaced-apart annular flanges 1092 so that the carriage may be moved distally or proximally along the B-B axis to adjust the depth of insertion of an airway device in a patient. Pressure applied to a second control or release button 1150 operatively connected to elongate, c-shaped cylindrical member 1102, and which extends through an aperture 1153 formed in the clamshell 1075. provides rotational movement of the cylindrical members 1102, 1104 and carriage member 1100 away from the annular flanges 1092. Once clear of the flanges, the carriage member may be moved upwardly or downwardly in a direction parallel to axis B-B by holding the release buttons 1147 and 1150 between two fingers. The adjustability in the position of the integrated carriage device provided by the system permits an individual installing an airway device in a patient to adjust the insertion depth of the airway device in response to the anatomical structure of a patient. A novel feature of this mechanism is that it allows vertical adjustment of the carriage member within the clamshell without having to open and remove the carriage member from the clamshell, an important safety feature to ensure the tube Is not extubated from the patient during vertical adjustment.

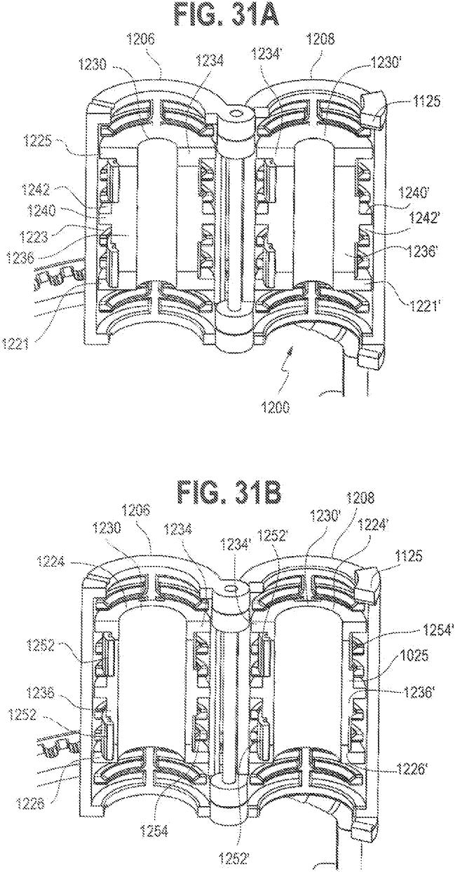

Referring now to FIGS. 30, 31.A-B and 32.A-C, another embodiment 1041 of the securing apparatus 1040 of FIG. 2 is illustrated which includes a tower structure or clamshell-type clamping member 1200 that having an individual grommet member 1205 adjustably positioned therein and adapted to releasably engage an airway device. Of similar construction to the tower structure of FIG. 26, the clamping member includes a pair of oppositely disposed pivotally interconnected c-shaped collars or clamshells 1206, 1208, each collar or clamshell having a length, first end and second ends 1210, 1210′ and 1212, 1212′, an inner surface 1214, 1214′ and an outer surface 1216, 1216′. Each of the clamshells has a pair of generally parallel extending edge surfaces 1218, 1218′ and 1220, 1220′ respectively, the edge surfaces and the corresponding c-shaped clamshell each defining an opposed, semi-cylindrically shaped cavity 1222, 1222′ about the axis B-B. As described above with respect to an alternate embodiment of FIG. 26, the inner surfaces 1214, 1214′ of the clamshells each include a plurality of substantially uniformly spaced-apart annular flanges 1092 positioned axially along the inner surface thereof and extending substantially inwardly therefrom, and a plurality of structural recesses 1094 positioned axially along the inner surface of the collar or clamshell intermediate an adjacent two of the plurality of substantially uniformly spaced-apart annular flanges.

The grommet 1205 of the embodiment of FIG. 30 is formed by a pair of elongate c-shaped cylindrical grommet halves or sections 1221, 1221′, each positioned within and operatively connected to a respective one of the c-shaped collars or clamshells 1206, 1208 and extending outwardly from the patient’s face coaxially with the longitudinal axis B-B of the clamping member. Referring to FIGS. 31.A, 31.B, 32.B and 32.C, each .of the elongate cylindrical members includes first and second ends 1224, 1226 and 1224′, 1226′ respectively and a body portion 1228, 1228′ having an inner surface 1230, 1230′, an outer surface 1232, 1232′, and a pair of generally parallel extending edge surfaces 1234, 1234′ and 1236, 1236′ respectively’ extending therebetween. The outer surface of each c-shaped section 1220, 1220′ includes at least one annular flange 1240, 1240′ and structural recess 1242, 1242′ extending radially outwardly from a respective outer surface 1232, 1232′ and adapted to operatively interact with one of the plurality of structural recesses formed intermediate the substantially uniformly spaced-apart annular flanges positioned axially along the inner surface of clamshells 1206, 1208.

In a closed configuration, the tower collars or clamshells 1206, 1208 are retained in locking engagement by a releasable latch mechanism or button shown as element 1125 in FIG. 32.A. The grommet sections are adapted to be retained in each respective clamshell following placing them therein when the collars or clamshells are in either an open or a closed position. By depressing the release button, the collars may be opened, thus disengaging each of the grommets from interacting engagement with the airway device, thereby permitting the individual performing the intubation to adjust the depth of insertion of the airway device in a patient without having to remove the securing apparatus from the patient’s face. Once the proper insertion depth has been realized, the clamshells and the grommets may be again closed about the airway device (or, in an alternate embodiment, the carriage member) to secure the system in position on the patient. In practice, the arrangement and interaction of the individual components allows the entire system to be used as a patient intubation aid. The two c-shaped grommet sections are also releasably secured to one another by a latch or other suitable interlocking feature 1250. In the embodiment shown, the interlocking feature is in the form of a curvilinear clasp 1252, 1252′ operatively connected to each edge 1236 and 1234′ respectively. Each clasp includes a plurality of teeth 1254, 1254′, the teeth being structured and arranged to selectively engage a rib or flange 1256, 1256′ extending along and radially outwardly from edges or surfaces 1234, 1236′. In addition, the inner surfaces of each c-shaped grommet section 1220, 1220′ may be coated with an adhesive material, by way of example and not of limitation, a pressure sensitive adhesive (PSA) adapted to adhesively engage the outer surface of an airway device. In yet another embodiment, the inner surfaces may be textured, for example, like the surface texturing found on a porcupine quill, to selectively prevent axial motion along the B-B axis of an airway device in one or both axial directions.

In operation the grommet members or sections 1221, 1221′ are interconnected in mating alignment within the clamping member 1200 thereby forming an aperture 1260 adapted to receive and secure an airway device 5. The embodiment of FIGS. 31.B and 32.B-32.Cdepicts a grommet adapted to receive and secure a large diameter airway device, for example, a device having an outer diameter of 6.0 mm. In the embodiment of FIG. 31.A, the two grommet sections are sized to form an aperture of sufficient size to receive a smaller diameter airway device, by way of example, an airway device having a body diameter of 3.0 mm. In accordance with the present invention, grommet sections of various sizes may be selected for use with and to secure airway devices having corresponding body diameters as required by the patient’s anatomy in any given situation. Moreover, as shown in FIG. 31.A, multiple individual grommet sections 1221, 1223. 1225 may be stacked within the tower structure 1200 without departing from the scope of the present invention.

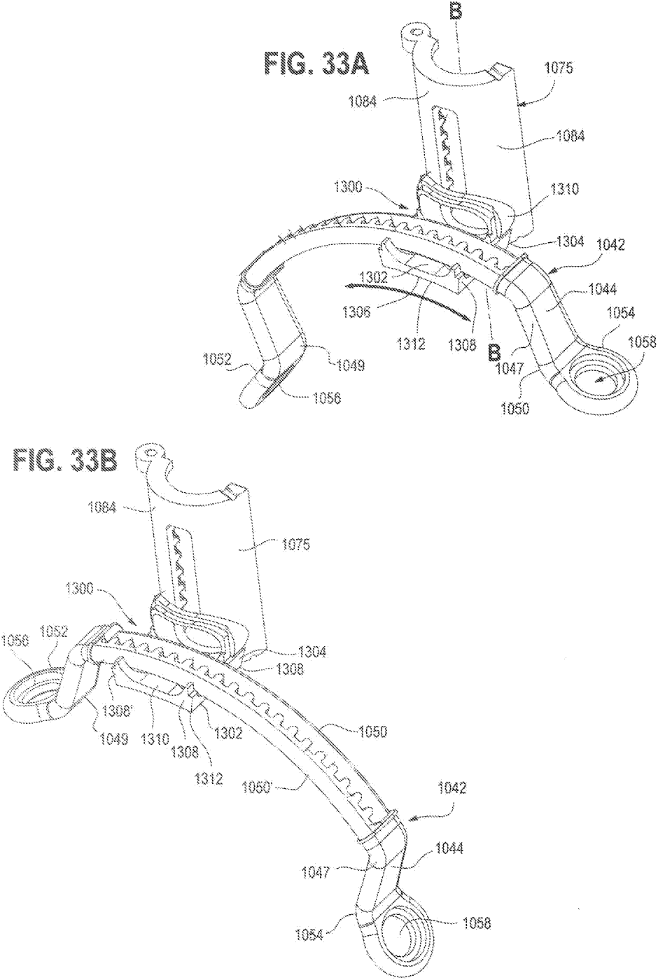

Referring to FIGS. 33.A through 34.B, the lateral position adjustment features of the present invention are specifically described. A bracket or mounting arm 1302 of an attachment apparatus 1300 is operatively connected to the outer surface 1084 of clamshell 1075 and extends therefrom in a direction substantially perpendicular to the longitudinal axis B-B. The mounting arm includes a first end 1304, a second end 1306, a pair of spaced apart side portions 1308, 1308′ and oppositely disposed top and bottom surfaces 1310, 1312 extending intermediate the ends and the side portions and cooperating therewith to form the mounting arm.

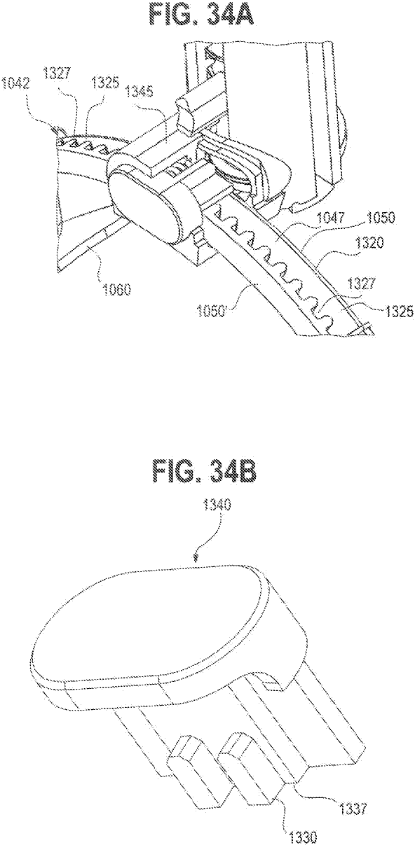

As shown more clearly in FIG. 34.A, a linear track 1320 is formed in the top surface 1047 of the bracket 1042 and includes a plurality of spaced apart teeth 1325 extending from the side portion 1050′ into the linear track in a direction generally perpendicular to the axis B-B and toward the clamshell 1075. Each of the plurality of teeth is separated from an adjacent one of the plurality of teeth by a space or recess 1327, the plurality of teeth and recesses being adapted to releasably engage a plurality of mating teeth formed on a stepped bottom surface 1337 of a release attachment or button 1340 (FIG. 34.B). The release button is structured and arranged to moveably fit over the second end 1306 of the mounting arm 1302 and be releasably secured thereto. A biasing mechanism 1345, for example, a compression spring is positioned between the release button and the top surface 1047 intermediate the first and second ends 1304, 1306 of the mounting arm and is adapted to maintain the teeth 1330 in releasable engagement with the plurality of teeth and spaces or recesses on the bridge member 1042. Pushing on the release button in a direction toward the clamshell 1075 compresses the spring and releases the teeth, whereby the lateral position of the tower structure 1070 may be adjusted on a patient in response to the patient’s anatomical features.

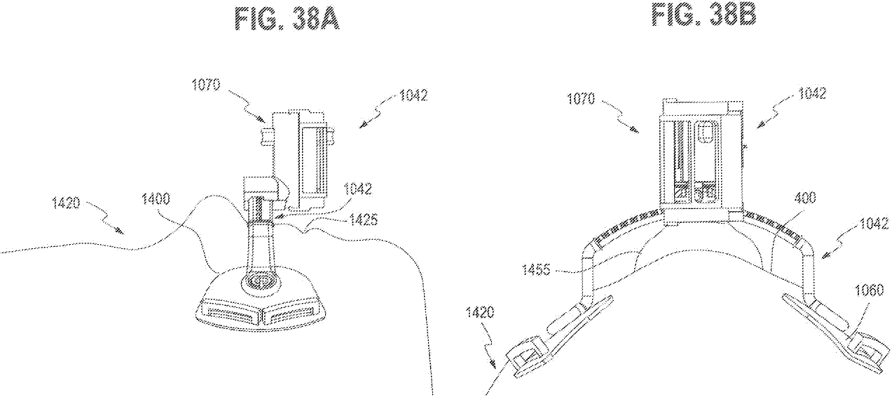

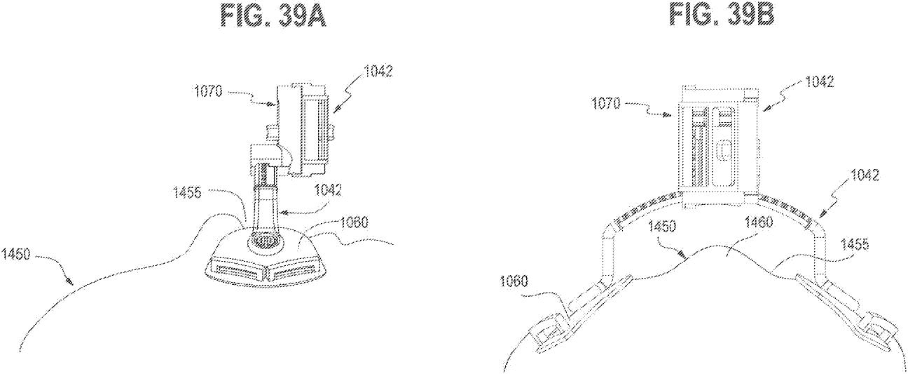

Portions of an embodiment 1042 of the airway stabilization system of the present invention configured for use in securing and stabilizing an airway device on a pediatric patient 1420 is shown in FIGS. 38.A and 38.Band on an infant 1450 in FIGS. 39.A and 39.B. FIGS. 38.A and 38.Billustrate the clamping system 1070 is mounted on bridge or supporting system 1042, which is positioned on the face 1400 of pediatric patient 1420 in the six- to eight-year-old age bracket. The system is installed on the face 1455 of infant patient 1450 in the age bracket of newborn to approximately three months of age. As evident from both the side and top views of the patients and the system, the structure of the bridge or frame 1042 and the attachment apparatus enables precise alignment of an intubation device with the patient’s mouth 1425 or 1460 and oral cavity and clearances between the apparatus and the patient’s face 1400 or 1450, respectively. The arrangement of the interacting components of the system permit its use over a broad range of facial geometries, adapted to be used with small or large diameter airway devices as described above with respect to the embodiments of FIGS. 28 and 29, as the situation dictates. The flexibility and adaptability of the system of the present invention is highly advantageous in emergency and field applications where immediate intubation and stabilization of the airway device may be required in life-threatening situations.

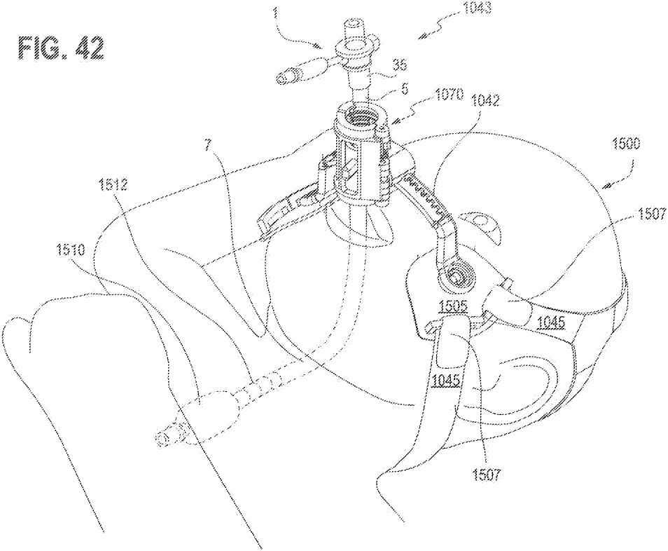

FIGS. 40-42 depict the installation of the airway stabilization system 1 of the present invention on an infant patient 1500 in accordance with an embodiment 1043. In this embodiment, a cheek piece or cushioning layer 1505 is provided for the comfort of the patient and includes a pair of apertures or slots 1507 formed therein, each adapted to receive a strap or headband securing device 1045 to maintain the airway device in its critical position in the patient and to prevent unintended or accidental extubation of the infant. Referring to FIG. 42, portions of an endotracheal tube 7 are shown positioned in the infant’s airway to better illustrate the positioning of an inflation balloon 1510 and depth indicator or location bands 1512 within the patient’s airway.

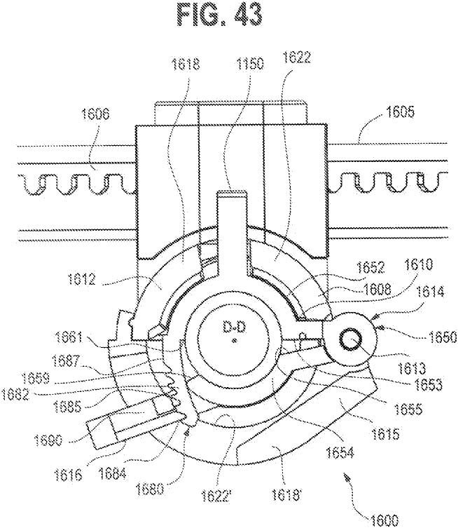

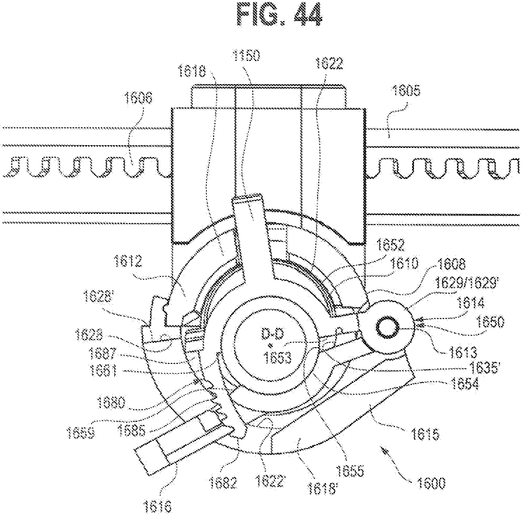

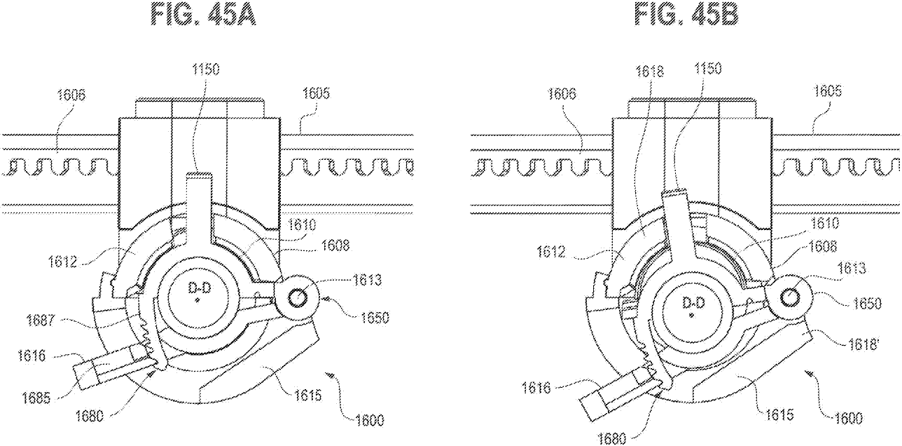

Referring now to FIGS. 43-46, portions of a securing apparatus 1600 are depicted in accordance with another embodiment of the present invention. The securing apparatus 1600 includes a supporting bridge or frame 1605 which may be secured to the patient’s face by a suitable attachment apparatus. The bridge or frame includes an outer surface 1606 and is preferably of unitary construction and in a generally symmetrical configuration contoured to permit it to conform to a patient’s face when it is secured in position. The securing apparatus 1600 includes a generally cylindrically shaped tower structure or clamshell-type clamping member 1608 operatively connected to the frame extending in a substantially perpendicular direction from the outer surface 1606 thereof along axis D-D. The clamping member is configured to interact in clamping engagement with the continuous sidewall of an airway device via a carriage member 1610 adjustably positioned in the tower structure to prevent clinically significant movement of the patient-end of the airway device with respect to the vocal cords of the patient. The clamping member includes a pair of oppositely disposed pivotally interconnected c-shaped collars or clamshells 1612, 1615, each collar or clamshell having a length, first end and second ends 1618, 1618′ and 1620, 1620′, an inner surface 1622, 1622′ and an outer surface 1624, 1624′, Each of the clamshells has a pair of generally parallel extending edge surfaces 1628, 1628′ and 1629, 1629′ respectively, the edge surfaces and the corresponding c-shaped clamshell each defining an opposed, semi-cylindrically shaped cavity 1635, 1635′ about the axis D-D. The inner surface 1622 of clamping member or collar 1612 includes a plurality of substantially uniformly spaced-apart annular flanges 1640 positioned axially along the inner surface thereof and extending substantially inwardly therefrom, and a plurality of structural recesses 1642 positioned axially along the inner surface of the collar or clamshell intermediate an adjacent two of the plurality of substantially uniformly spaced-apart annular flanges.

The airway stabilization system 1600 of the embodiment of FIGS. 43-46 further includes an integrated carriage member or collar device 1650. Similar in configuration and operation to the carriage member or collar of the embodiment of FIG. 26 et seq., the carriage member/collar 1650 is formed by a pair of pivotally interconnected elongate c-shaped cylindrical members 1652, 1654, each positioned within and operatively connected to a respective one of the tower’s c-shaped collars or clamshells 1612, 1615 and extending outwardly from the patient’s face coaxially with the longitudinal axis D-D of the clamping member.

In operation the collars or clamshells 1612, 1615 and c-shaped cylindrical members 1652, 1654 are pivotally interconnected, for example, by a pin 1613 which extends through a plurality of hinge members shown generally at 1614 operatively connected to edges 1629 and 1629′ of the clamshells and to edges 1653 and 1655 of the c-shaped cylindrical members 1652 and 1654, respectively. A handle or operating arm 1616 extends radially outwardly from edge 1659 of collar 1615 and is adapted to facilitate opening and closing of the clamshells. In the instant embodiment, the operating arm has a length structured an arranged to leverage minimal rotational assembly force to the apparatus to facilitate installation and adjustment of the system on a patient. The collars may be retained in locking engagement by a releasable latch mechanism (not shown) of essentially the same construction and operation as the latch mechanism illustrated as element 1125 in FIGS. 28.A, 28.Band also in FIG. 32.A.

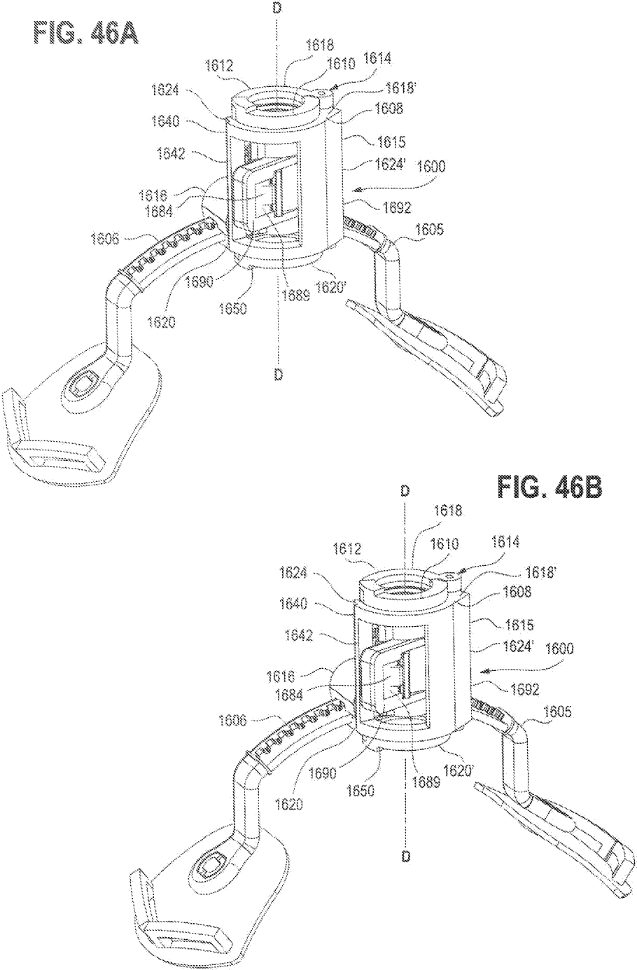

As described above with respect to alternate embodiments of the present invention, the c-shaped collars are adjustably latched together in response to the varying sizes of airway devices used to intubate the patient by a latch or other suitable interlocking feature 1680. In the embodiment shown, the interlocking feature is in the form of a ratchet having a curvilinear member 1682 operatively connected to edge 1661. Member 1680 includes a plurality of ratchet teeth 1685 formed integrally on or operatively secured to an outer surface 1687 thereof. As best shown in FIGS. 46.A and 46.B, the curvilinear member 1682 extends through an aperture or window 1689 formed in operating arm 1616, the ratchet teeth being structured and arranged to selectively engage a mating locking member 1684 positioned in the aperture 1689. By way of example and not of limitation, FIGS. 43 and 46.Aillustrate a locking member in the form of an angled snap 1690 which is adapted via internal spring forces created by its shape and material properties to be urged into operative engagement with the ratchet teeth, thus preventing unintentional opening of the carriage when an airway device is removed from a patient’s airway. FIG. 46.8 depicts yet another embodiment of a locking member in the form of a zip tie snap 1692 which retains the operating arm in locking engagement with the curvilinear member 1682 as the c-shaped cylindrical members or collars are selectively tightened circumferentially around an airway device in response to its outer diameter.

While only selected embodiments have been chosen to illustrate the present invention, it will be apparent to those skilled in the art from this disclosure that various changes and modifications can be made herein without departing from the scope of the invention as defined in the appended claims. Furthermore, the foregoing descriptions of the embodiments according to the present invention are provided for illustration only, and not for the purpose of limiting the invention as defined by the appended claims and their equivalents.

What is claimed is:

1. A complete airway stabilization system for securing any airway device that may be used with human patients or with animal patients in veterinary applications to maintain an airway in human or animal patients having anatomical and facial geometries of various sizes, the patient having a head, a face, a chin, a chest, a mouth, an oral cavity, vocal cords or larynx, a thoracic area, a trachea having a length and forming an airway in the patient, and a carina defining a point at which the trachea separates into a left and a right bronchial tube, the airway system comprising:
an airway device having a flexible elongate body having a longitudinal axis, the flexible body being adapted to conform to a patient’s trachea after it is installed in a preselected position in the patient, the flexible body including a continuous sidewall having outer and inner surfaces extending between a proximal (patient-end) and a distal (machine-end) portion thereof, thereby forming a hollow conduit or airway;
a support member or frame secured to the patient;
a securing device or tower structure adjustably secured to the frame or support member and extending outwardly therefrom along the longitudinal axis of the airway device in a direction away from a patient’s face; and
a carriage member or collar adjustably positioned in the tower structure and adapted to receive the airway device in securing engagement therewith, the carriage member being adapted to secure airway devices of different sizes and to cooperate with the securing device to maintain the airway device in the preselected position in the patient and to prevent movement thereof as a result of multidirectional forces being applied to the airway device.

2. The airway stabilization system of claim 1 wherein the securing device or tower structure member includes a pair of oppositely disposed pivotally interconnected c-shaped collars or clamshells, each collar or clamshell having a first end and a second end and a body portion extending therebetween, the body portion having an inner surface and an outer surface, the inner surface of at least one of the c-shaped collars or clamshells including a plurality of uniformly spaced-apart annular flanges positioned axially along the inner surface of the body portion and extending inwardly therefrom, and a plurality of structural recesses positioned axially along the inner surface of the body portion intermediate an adjacent two of the plurality of uniformly spaced-apart annular flanges and structural recesses of the clamshells.

3. The airway stabilization system of claim 2 wherein the inner surfaces of each of the pair of oppositely disposed pivotally interconnected c-shaped collars or clamshells includes a plurality of uniformly spaced-apart annular flanges positioned axially along the inner surface of the body portion and extending inwardly therefrom, and a plurality of structural recesses positioned axially along the inner surface of the body portion intermediate an adjacent two of the plurality of uniformly spaced-apart annular flanges and structural recesses of the clamshells.

4. The airway stabilization system of claim 3 wherein the carriage member or collar includes a pair of pivotally interconnected elongate c-shaped cylindrical members, each cylindrical member being positioned within and operatively connected to a respective one of the c-shaped collars or clamshells and extending outwardly from the patient’s face coaxially with the longitudinal axis of the airway device.

5. The airway stabilization system of claim 4 wherein each of the c-shaped elongate cylindrical members includes first and second ends and a body portion having an inner surface and an outer surface extending therebetween, the outer surface of at least one of the pair of cylindrical members including at least one annular flange and structural recess extending radially outwardly from the outer surface and being adapted to operatively interact with one of the plurality of structural recesses formed intermediate the substantially uniformly spaced-apart annular flanges positioned axially along the inner surface of the at least one of the clamshells.

6. The airway stabilization system of claim 5 wherein the outer surface of at least one of the pair of cylindrical members includes at least one annular flange and structural recess extending radially outwardly from the outer surface and adapted to operatively and releasably interact with one of the plurality of structural recesses formed intermediate the substantially uniformly spaced-apart annular flanges positioned axially along the inner surface of the at least one of the clamshells, thereby providing vertical adjustment of the airway device in the patient’s trachea.

7. The airway stabilization system of claim 5 wherein the inner surface of at least one of the pair of cylindrical members is coated with an adhesive material.

8. The airway stabilization system of claim 7 wherein the adhesive material is a pressure sensitive adhesive (PSA) adapted to adhesively engage the outer surface of the airway device.

9. The airway stabilization system of claim 7 wherein the adhesive material is a permanent bonding agent adapted to be applied or injected into an interface intermediate the outer surface of the airway device and the inner surface of the at least one of the pair of cylindrical members.

10. The airway stabilization system of claim 3 wherein the carriage member or collar comprises one or more semi-cylindrically or c-shaped grommets, each of the grommets being positioned within and operatively connected to a respective one of the c-shaped collars or clamshells of the tower structure and extending outwardly from the patient’s face coaxially with the longitudinal axis of the airway device.

11. The airway stabilization system of claim 10 wherein each c-shaped grommet has first and second ends and a body portion having an inner surface and an outer surface extending therebetween, the outer surface of each grommet including at least one annular flange and at least one structural recess extending radially outwardly from the outer surface and adapted to operatively interact with one of the plurality of structural recesses formed intermediate the uniformly spaced-apart annular flanges positioned axially along the inner surface of at least one of the clamshells.

12. The airway stabilization system of claim 10 wherein the inner surface of at least one of the grommets is coated with an adhesive material.

13. The airway stabilization system of claim 12 wherein the securing device or tower structure includes an adjustable ratchet mechanism adapted to releasably clamp airway devices of different flexible body sizes.

14. The airway stabilization system of claim 13 wherein the carriage member includes a release mechanism adapted to permit selective adjustment of the position of an airway device with respect to a patient’s vocal cords.

15. The airway stabilization system of claim 14 wherein the securing device or tower structure includes a release mechanism adapted to selectively position the carriage member within the clamping member.

16. The airway stabilization system of claim 1 wherein the airway stabilization system includes a lateral position adjustment mechanism adapted to laterally adjust the position of the tower structure on the support member.

17. The airway stabilization system of claim 16 wherein the support member includes a body, an upper or outer surface facing away from the patient and a lower or bottom surface facing toward the patient, the upper and lower surfaces being interconnected by a pair of oppositely disposed, spaced apart side portions and first and second oppositely disposed end portions.

18. The airway stabilization system of claim 17 wherein the support member includes a cheek pad or layer of cushioning material releasably secured to a lower surface of each of the first and second oppositely disposed end portions.

19. The airway stabilization system of claim 19 wherein the cheek pad or layer of cushioning material is a hydrocolloid absorbent, waterproof material, or a silicone gel pad.

20. The airway stabilization system of claim 20 wherein each cheek pad includes a skin contact adhesive layer having a perforated pattern whereby moisture is wicked to the cushioning material.
